SEQUENCE LISTING (217 sequences)

SEQ ID NO: 1 - protein, 13 aa, Homo sapiens
QWTQQHLDAADLR

SEQ ID NO: 2 - protein, 15 aa, Homo sapiens
QASTLADLDSGNMEK

SEQ ID NO: 3 - protein, 17 aa, Homo sapiens
NLRFEINCIPNLIEYVK

SEQ ID NO: 4 - protein, 19 aa, Homo sapiens
KEVGDVSILINNAGIVTGK

SEQ ID NO: 5 - protein, 17 aa, Homo sapiens
NNNTDLMILKNTKDAVR

SEQ ID NO: 6 - protein, 17 aa, Homo sapiens
GSIEILRDDVVVAILGK

SEQ ID NO: 7 - protein, 24 aa, Homo sapiens
TASALFAGFRALGLFSNDIPHVVR

SEQ ID NO: 8 - protein, 10 aa, Homo sapiens
ERVTPPISER

SEQ ID NO: 9 - protein, 14 aa, Homo sapiens
AAGSDADHLREQQR

SEQ ID NO: 10 - protein, 17 aa, Homo sapiens
PNKVNFGTDFLTAIKNR

SEQ ID NO: 11 - protein, 13 aa, Homo sapiens
TPKCTVLLSLIQR

SEQ ID NO: 12 - protein, 11 aa, Homo sapiens
DTLYLQMNSLR

SEQ ID NO: 13 - protein, 17 aa, Homo sapiens
TLKLKYAELAALEFPPK

SEQ ID NO: 14 - protein, 14 aa, Homo sapiens
SPAQAELSYLNKAK

SEQ ID NO: 15 - protein, 11 aa, Homo sapiens
RLGVTLVGHQK

SEQ ID NO: 16 - protein, 18 aa, Homo sapiens
EVELYVTEIEKEKEEAEK

SEQ ID NO: 17 - protein, 21 aa, Homo sapiens
DSLTLHTKPEPLEGPALSHSV

SEQ ID NO: 18 - protein, 25 aa, Homo sapiens
EDVDPHNGADDVFSSSGSLGKASEK

SEQ ID NO: 19 - protein, 16 aa, Homo sapiens
EVGNLLLENSQLLETK

SEQ ID NO: 20 - protein, 13 aa, Homo sapiens
SDLAVPSELALLK

SEQ ID NO: 21 - protein, 21 aa, Homo sapiens
SPPLLESPDATRESMVKLSSK

SEQ ID NO: 22 - protein, 17 aa, Homo sapiens
EACIVEALGIQTLTNQK

SEQ ID NO: 23 - protein, 14 aa, Homo sapiens
MAAGEPRSLLFFQK

SEQ ID NO: 24 - protein, 14 aa, Homo sapiens
MVFHITTGSQEFDK

SEQ ID NO: 25 - protein, 16 aa, Homo sapiens
FIGSLHSYSFSSKHTR

SEQ ID NO: 26 - protein, 24 aa, Homo sapiens
PAVWLSWKVSGPAAPAQSYTALFR

SEQ ID NO: 27 - protein, 13 aa, Homo sapiens
YIGIRMMSLTSSK

SEQ ID NO: 28 - protein, 16 aa, Homo sapiens
NLYLAWNCYFNKVCEK

SEQ ID NO: 29 - protein, 15 aa, Homo sapiens
VEMANLLNLTERQIK

SEQ ID NO: 30 - protein, 30 aa, Homo sapiens
LVEVIPEGAMLRLGMTNPPYILEHLEEMAK

SEQ ID NO: 31 - protein, 24 aa, Homo sapiens
PAASPNTTSSRGQTVHPPCSSKLR

SEQ ID NO: 32 - protein, 21 aa, Homo sapiens
NRLTDYYEPLLKNNSTAYSTR

SEQ ID NO: 33 - protein, 15 aa, Homo sapiens
FAMPSSTPLYHDAIK

SEQ ID NO: 34 - protein, 19 aa, Homo sapiens
SCDASLAELGDDKSGDASR

SEQ ID NO: 35 - protein, 9 aa, Homo sapiens
VTISVDTSK

SEQ ID NO: 36 - protein, 14 aa, Homo sapiens
MDINTYNNQLHLQR

SEQ ID NO: 37 - protein, 11 aa, Homo sapiens
AEDTALYYCAK

SEQ ID NO: 38 - protein, 24 aa, Homo sapiens
SHLTVCFLPSVPFLILVSTLATAK

SEQ ID NO: 39 - protein, 19 aa, Homo sapiens
ELSENINAVTLMKEELKEK

SEQ ID NO: 40 - protein, 23 aa, Homo sapiens
PGQNSCRDSDSESASGESKGFHR

SEQ ID NO: 41 - protein, 16 aa, Homo sapiens
DANSVITYQLTGGNTR

SEQ ID NO: 42 - protein, 22 aa, Homo sapiens
DEIQCVIATIAFGMGINKADIR

SEQ ID NO: 43 - protein, 24 aa, Homo sapiens
AVIKNRNTTYIVIGAVQPNDISNK

SEQ ID NO: 44 - protein, 27 aa, Homo sapiens
VMQLQYENRVLMSNMQRYDLASHLGIR

SEQ ID NO: 45 - protein, 21 aa, Homo sapiens
GRILSGNFFLLKENYENYFVK

SEQ ID NO: 46 - protein, 10 aa, Homo sapiens
DRDGRPQVVR

SEQ ID NO: 47 - protein, 15 aa, Homo sapiens
NVEVTVSVYDEDGKR

SEQ ID NO: 48 - protein, 22 aa, Homo sapiens
KGQEGESCLRTFDCGPGLCCAR

SEQ ID NO: 49 - protein, 12 aa, Homo sapiens
TCCHIMLSQEEK

SEQ ID NO: 50 - protein, 19 aa, Homo sapiens
CKDGLGEYTCTCLEGFEGK

SEQ ID NO: 51 - protein, 11 aa, Homo sapiens
AVIFITKRGLK

SEQ ID NO: 52 - protein, 18 aa, Homo sapiens
QENEKPLPENMDAFEKVR

SEQ ID NO: 53 - protein, 19 aa, Homo sapiens
AKEFIIGTGWEEAVQGWGR

SEQ ID NO: 54 - protein, 16 aa, Homo sapiens
LPKGMQARAPSQYSTR

SEQ ID NO: 55 - protein, 15 aa, Homo sapiens
EEAREEFLSASPEPR

SEQ ID NO: 56 - protein, 11 aa, Homo sapiens
LDTSQWPLLLK

SEQ ID NO: 57 - protein, 19 aa, Homo sapiens
EEAPSLRPAPPPISGGGYR

SEQ ID NO: 58 - protein, 25 aa, Homo sapiens
DWSHYFKIIEDLRAQIFANTVDNAR

SEQ ID NO: 59 - protein, 8 aa, Homo sapiens
FLNVLSPR

SEQ ID NO: 60 - protein, 11 aa, Homo sapiens
QNCKGDSQPNK

SEQ ID NO: 61 - protein, 18 aa, Homo sapiens
VVTLAQHISEVITQDYTR

SEQ ID NO: 62 - protein, 16 aa, Homo sapiens
ICDQWDNLGALTQKRR

SEQ ID NO: 63 - protein, 18 aa, Homo sapiens
LGQSSMLGEQGARIQSVK

SEQ ID NO: 64 - protein, 21 aa, Homo sapiens
SCQSGEDEEEDEDKEKTFEEK

SEQ ID NO: 65 - protein, 17 aa, Homo sapiens
ALSDVTDEEEVQVDPRK

SEQ ID NO: 66 - protein, 17 aa, Homo sapiens
DVKDTVTEILDTKVFSY

SEQ ID NO: 67 - protein, 13 aa, Homo sapiens
CFVESLSSVETLK

SEQ ID NO: 68 - protein, 12 aa, Homo sapiens
PCWELKKIMILK

SEQ ID NO: 69 - protein, 26 aa, Homo sapiens
DIGVPMTSVRLPCYFENLLSHFLPQK

SEQ ID NO: 70 - protein, 20 aa, Homo sapiens
ADSVANQGTKVEGITNQGKK

SEQ ID NO: 71 - protein, 11 aa, Homo sapiens
AENTAVYYCAR

SEQ ID NO: 72 - protein, 22 aa, Homo sapiens
EPLIIHWAASPQRIEECVLSGK

SEQ ID NO: 73 - protein, 13 aa, Homo sapiens
MDVDKELGMESVK

SEQ ID NO: 74 - protein, 20 aa, Homo sapiens
DVAGARGAPPAWGQAPSPRR

SEQ ID NO: 75 - protein, 15 aa, Homo sapiens
ETYGHLGALGCAGPK

SEQ ID NO: 76 - protein, 11 aa, Homo sapiens
KKILDADIQLK

SEQ ID NO: 77 - protein, 30 aa, Homo sapiens
PKMLVISGGDGYEDFRLSSGGGSSSETVGR

SEQ ID NO: 78 - protein, 11 aa, Homo sapiens
EPETQPVCFFR

SEQ ID NO: 79 - protein, 23 aa, Homo sapiens
GPLLAACSSESRELCFVALCHVR

SEQ ID NO: 80 - protein, 22 aa, Homo sapiens
GASSSTNDASVPTTKEFETLIK

SEQ ID NO: 81 - protein, 14 aa, Homo sapiens
NKPGVYTKVYNYVK

SEQ ID NO: 82 - protein, 25 aa, Homo sapiens
GTLMELGISPIVTSGLIMQLLAGAK

SEQ ID NO: 83 - protein, 14 aa, Homo sapiens
ENKFPLSNQNMLLR

SEQ ID NO: 84 - protein, 15 aa, Homo sapiens
GRGSQGNLIFGKGTK

SEQ ID NO: 85 - protein, 8 aa, Homo sapiens
ISSTSTDR

SEQ ID NO: 86 - protein, 8 aa, Homo sapiens
QTVEVLDK

SEQ ID NO: 87 - protein, 17 aa, Homo sapiens
QVMAALVVSGAAEQGGR

SEQ ID NO: 88 - protein, 22 aa, Homo sapiens
GEIDASVPELEGDLRGPQVDVK

SEQ ID NO: 89 - protein, 19 aa, Homo sapiens
YLLPGSTQFFLRTPTYNLK

SEQ ID NO: 90 - protein, 11 aa, Homo sapiens
ESKKLLTIILK

SEQ ID NO: 91 - protein, 8 aa, Homo sapiens
EIISEVQR

SEQ ID NO: 92 - protein, 14 aa, Homo sapiens
QALMVTHKELATIK

SEQ ID NO: 93 - protein, 25 aa, Homo sapiens
YWGEIPISSSQTNRSSFDLLPREFR

SEQ ID NO: 94 - protein, 22 aa, Homo sapiens
FHLATAEWIAEPVRQKIAITHK

SEQ ID NO: 95 - protein, 14 aa, Homo sapiens
EVHSEIIQVEATDK

SEQ ID NO: 96 - protein, 22 aa, Homo sapiens
AGFAFEIPMKITWVSTVERGQK

SEQ ID NO: 97 - protein, 20 aa, Homo sapiens
VIIISILQQVMANTLEINGK

SEQ ID NO: 98 - protein, 20 aa, Homo sapiens
FELQDSGSSLLPKEIVKVEK

SEQ ID NO: 99 - protein, 15 aa, Homo sapiens
ELKALEEALRASQEK

SEQ ID NO: 100 - protein, 18 aa, Homo sapiens
GCWGLSCQLLEHAVRLCR

SEQ ID NO: 101 - protein, 7 aa, Homo sapiens
SLLEEER

SEQ ID NO: 102 - protein, 11 aa, Homo sapiens
LEYYESEKKWR

SEQ ID NO: 103 - protein, 7 aa, Homo sapiens
SNTLRER

SEQ ID NO: 104 - protein, 12 aa, Homo sapiens
QLQEFIPNIKDR

SEQ ID NO: 105 - protein, 11 aa, Homo sapiens
WEILWSERPTK

SEQ ID NO: 106 - protein, 13 aa, Homo sapiens
APQTGIVDECCFR

SEQ ID NO: 107 - protein, 16 aa, Homo sapiens
RLIYGATSLQSGVPSR

SEQ ID NO: 108 - protein, 20 aa, Homo sapiens
NVVVACENGLPVHLDQSIFR

SEQ ID NO: 109 - protein, 20 aa, Homo sapiens
LNTIPLFVQLLYSSVENIQR

SEQ ID NO: 110 - protein, 16 aa, Homo sapiens
DIKLENLMLDKDGHIK

SEQ ID NO: 111 - protein, 21 aa, Homo sapiens
VFDDLTLHKTDLEIQIEELNK

SEQ ID NO: 112 - protein, 8 aa, Homo sapiens
SSDANLYR

SEQ ID NO: 113 - protein, 10 aa, Homo sapiens
ISSVPAINNR

SEQ ID NO: 114 - protein, 21 aa, Homo sapiens
PLIACVEKQLLGEHLTAILQK

SEQ ID NO: 115 - protein, 14 aa, Homo sapiens
ESRNELQITSLNHK

SEQ ID NO: 116 - protein, 21 aa, Homo sapiens
SLQLSVDNVTVEGQMAGAHMR

SEQ ID NO: 117 - protein, 12 aa, Homo sapiens
SILDQSISSFMR

SEQ ID NO: 118 - protein, 12 aa, Homo sapiens
IGVTVLSRIHAR

SEQ ID NO: 119 - protein, 18 aa, Homo sapiens
EFYSEILDKKFTVTVTMR

SEQ ID NO: 120 - protein, 13 aa, Homo sapiens
TVVQSCGHSLETK

SEQ ID NO: 121 - protein, 14 aa, Homo sapiens
LKDDLFLGVKDNPK

SEQ ID NO: 122 - protein, 20 aa, Homo sapiens
QATDGSLASILSSLSHDEKK

SEQ ID NO: 123 - protein, 11 aa, Homo sapiens
FNIFYPDLIDK

SEQ ID NO: 124 - protein, 18 aa, Homo sapiens
LTLARSLVLLDDLTKAEK

SEQ ID NO: 125 - protein, 20 aa, Homo sapiens
VCPFKSEKVVVEDTVPIIPK

SEQ ID NO: 126 - protein, 18 aa, Homo sapiens
QFHEAWSKLMEWLEESEK

SEQ ID NO: 127 - protein, 23 aa, Homo sapiens
YCVQLSQIQGLIGSVEEQLAQLR

SEQ ID NO: 128 - protein, 24 aa, Homo sapiens
TSVQQTLPLRHPLPTLTRLYLASR

SEQ ID NO: 129 - protein, 14 aa, Homo sapiens
LDSGDLLQQAQEIR

SEQ ID NO: 130 - protein, 20 aa, Homo sapiens
MARIILQDEDVTTKIDNDWK

SEQ ID NO: 131 - protein, 13 aa, Homo sapiens
EVEVEYSTVASPR

SEQ ID NO: 132 - protein, 17 aa, Homo sapiens
ASTPNGYDNGIIWATWK

SEQ ID NO: 133 - protein, 14 aa, Homo sapiens
DFHINLFQVLPWLK

SEQ ID NO: 134 - protein, 17 aa, Homo sapiens
TCPKPDDLPFSTVVPLK

SEQ ID NO: 135 - protein, 11 aa, Homo sapiens
AENAVECFQTK

SEQ ID NO: 136 - protein, 11 aa, Homo sapiens
LSCAASGFTFR

SEQ ID NO: 137 - protein, 27 aa, Homo sapiens
GSSGGGCFGGSSGGYGGLGGFGGGSFR

SEQ ID NO: 138 - protein, 14 aa, Homo sapiens
KWIYHLTEGSTDLR

SEQ ID NO: 139 - protein, 15 aa, Homo sapiens
EVQGFESATFLGYFK

SEQ ID NO: 140 - protein, 12 aa, Homo sapiens
SSEDPNEDIVER

SEQ ID NO: 141 - protein, 15 aa, Homo sapiens
YNSQNQSNNQFVLYR

SEQ ID NO: 142 - protein, 17 aa, Homo sapiens
LYGAQFHPEVGLTENGK

SEQ ID NO: 143 - protein, 18 aa, Homo sapiens
LVAGQQQVLQQLAEEEPK

SEQ ID NO: 144 - protein, 16 aa, Homo sapiens
QAPDTSDGSCTELPFK

SEQ ID NO: 145 - protein, 14 aa, Homo sapiens
IQTLDQNVEYNGCR

SEQ ID NO: 146 - protein, 7 aa, Homo sapiens
KELQEKR

SEQ ID NO: 147 - protein, 20 aa, Homo sapiens
REEAPSLRPAPPPISGGGYR

SEQ ID NO: 148 - protein, 8 aa, Homo sapiens
FLNVLSPR

SEQ ID NO: 149 - protein, 14 aa, Homo sapiens
LTEDNKKLEIDINK

SEQ ID NO: 150 - protein, 14 aa, Homo sapiens
MDLEENPDEQSEIR

SEQ ID NO: 151 - protein, 7 aa, Homo sapiens
EKGENDK

SEQ ID NO: 152 - protein, 11 aa, Homo sapiens
QNLRETQKFFR

SEQ ID NO: 153 - protein, 19 aa, Homo sapiens
ELPPELLAEIESTMPLCER

SEQ ID NO: 154 - protein, 23 aa, Homo sapiens
MVWRLVLLALWVWPSTQAGHQDK

SEQ ID NO: 155 - protein, 17 aa, Homo sapiens
EQLGEFYEALDCLCIPR

SEQ ID NO: 156 - protein, 14 aa, Homo sapiens
VELEVPQLCSFILK

SEQ ID NO: 157 - protein, 11 aa, Homo sapiens
APEPDLMSPTR

SEQ ID NO: 158 - protein, 13 aa, Homo sapiens
EVEVEYSTVASPR

SEQ ID NO: 159 - protein, 14 aa, Homo sapiens
SYGSVLLSAEEFQK

SEQ ID NO: 160 - protein, 12 aa, Homo sapiens
DINDHSPTFLDK

SEQ ID NO: 161 - protein, 10 aa, Homo sapiens
LNKPADFIGK

SEQ ID NO: 162 - protein, 22 aa, Homo sapiens
FNPICKASGWPLPTNEEMTLVK

SEQ ID NO: 163 - protein, 14 aa, Homo sapiens
VLTPYCYTIDVEIK

SEQ ID NO: 164 - protein, 18 aa, Homo sapiens
LIARYAARLAAEAGNVTR

SEQ ID NO: 165 - protein, 19 aa, Homo sapiens
SCDASLAELGDDKSGDASR

SEQ ID NO: 166 - protein, 10 aa, Homo sapiens
VYEMYVTVHR

SEQ ID NO: 167 - protein, 15 aa, Homo sapiens
ILSIDRNLRNQLFNR

SEQ ID NO: 168 - protein, 10 aa, Homo sapiens
YVGTGGIDQK

SEQ ID NO: 169 - protein, 10 aa, Homo sapiens
NMPSSKDTRK

SEQ ID NO: 170 - protein, 10 aa, Homo sapiens
RAIIEDLEPR

SEQ ID NO: 171 - protein, 14 aa, Homo sapiens
KDAFLYQRTLQFIR

SEQ ID NO: 172 - protein, 18 aa, Homo sapiens
EFYSEILDKKFTVTVTMR

SEQ ID NO: 173 - protein, 11 aa, Homo sapiens
WLSSPVFSLRR

SEQ ID NO: 174 - protein, 11 aa, Homo sapiens
AVIFITKRGLK

SEQ ID NO: 175 - protein, 30 aa, Homo sapiens
VPTPQAIRAAQGLLACGVAQGALRSNYVLK

SEQ ID NO: 176 - protein, 12 aa, Homo sapiens
NQANIHAVDNFK

SEQ ID NO: 177 - protein, 10 aa, Homo sapiens
VLSPYDLTHK

SEQ ID NO: 178 - protein, 12 aa, Homo sapiens
NNKGPVKVVVGK

SEQ ID NO: 179 - protein, 8 aa, Homo sapiens
RGANVNLR

SEQ ID NO: 180 - protein, 16 aa, Homo sapiens
SEQPTLVTASKDGYFK

SEQ ID NO: 181 - protein, 17 aa, Homo sapiens
VFKMLLGDEKQSIVQAK

SEQ ID NO: 182 - protein, 13 aa, Homo sapiens
GADPPPPPSRTGR

SEQ ID NO: 183 - protein, 17 aa, Homo sapiens
ENKNVKDPVEIQTLVNK

SEQ ID NO: 184 - protein, 13 aa, Homo sapiens
FNDEDIEKMEGKK

SEQ ID NO: 185 - protein, 20 aa, Homo sapiens
ADQEGAREIVCNVTLGGERR

SEQ ID NO: 186 - protein, 16 aa, Homo sapiens
AGECVTAGGLGGARRR

SEQ ID NO: 187 - protein, 20 aa, Homo sapiens
QVSQPALVIPPQPPTTGPPR

SEQ ID NO: 188 - protein, 12 aa, Homo sapiens
HLLTIAGWKHEK

SEQ ID NO: 189 - protein, 13 aa, Homo sapiens
APQTGIVDECCFR

SEQ ID NO: 190 - protein, 13 aa, Homo sapiens
FKPMQRHLFLHEK

SEQ ID NO: 191 - protein, 12 aa, Homo sapiens
VRASQELEMSLK

SEQ ID NO: 192 - protein, 16 aa, Homo sapiens
EKLSLEPVLPARNPNR

SEQ ID NO: 193 - protein, 17 aa, Homo sapiens
EDPDFLGAFLGELLPSR

SEQ ID NO: 194 - protein, 13 aa, Homo sapiens
VMSDGPVKPEGAK

SEQ ID NO: 195 - protein, 18 aa, Homo sapiens
DSGGQTSAGCPSGWLGTR

SEQ ID NO: 196 - protein, 11 aa, Homo sapiens
YLAIVHPLKPR

SEQ ID NO: 197 - protein, 13 aa, Homo sapiens
QAACLEQIREMEK

SEQ ID NO: 198 - protein, 24 aa, Homo sapiens
LSALPPEGGKPYLESFKLSLDNTK

SEQ ID NO: 199 - protein, 8 aa, Homo sapiens
QTVEVLDK

SEQ ID NO: 200 - protein, 11 aa, Homo sapiens
TTWMKADEVEK

SEQ ID NO: 201 - protein, 11 aa, Homo sapiens
FDLKQWLSATK

SEQ ID NO: 202 - protein, 17 aa, Homo sapiens
FPGVGVLPGVPTGAGVK

SEQ ID NO: 203 - protein, 18 aa, Homo sapiens
YAQICLWIHGNDANCAYG

SEQ ID NO: 204 - protein, 15 aa, Homo sapiens
CLRWGGGYEGENLMK

SEQ ID NO: 205 - protein, 12 aa, Homo sapiens
MIMILVLLSLGR

SEQ ID NO: 206 - protein, 13 aa, Homo sapiens
GSDLPPDPHNCGK

SEQ ID NO: 207 - protein, 7 aa, Homo sapiens
SLLEEER

SEQ ID NO: 208 - protein, 14 aa, Homo sapiens
ARRFLWNLILDLIK

SEQ ID NO: 209 - protein, 8 aa, Homo sapiens
RFNVAITR

SEQ ID NO: 210 - protein, 13 aa, Homo sapiens
PLAIAKQASFSSK

SEQ ID NO: 211 - protein, 15 aa, Homo sapiens
SMSCQEFITNLNGLR

SEQ ID NO: 212 - protein, 16 aa, Homo sapiens
EKEQEAGGVSYWDIGK

SEQ ID NO: 213 - protein, 18 aa, Homo sapiens
RQRGDAGSPWGCPLCPDR

SEQ ID NO: 214 - protein, 14 aa, Homo sapiens
RNPEDVQEIIVWKR

SEQ ID NO: 215 - protein, 8 aa, Homo sapiens
LAESEGTK

SEQ ID NO: 216 - protein, 17 aa, Homo sapiens
FLNLQNEHEKALGTWKR

SEQ ID NO: 217 - protein, 15 aa, Homo sapiens
TCLLNETGDEPFQYK

(19) United States
(12) Patent Application Publication
(10) Pub. No.: US 2010/0105087 A1
(43) Pub. Date: Apr. 29, 2010

(54) BIOMARKERS FOR BREAST CANCER

(75) Inventors: Emanuel F. Petricoin, Gainesville, VA (US); Wediong Zhou, Manassas, VA (US); Serena Camerini, Napoli (IT); Maria Letizia Polci, Chieti (IT); Lance Liotta, Bethesda, MD (US)

Correspondence Address: FOLEY AND LARDNER LLP;SUITE 500, 3000 K STREET NW, WASHINGTON, DC, 20007, US

(73) Assignee: George Mason Intellectual Properties, Inc

(21) Appl. No.: 12/446,936

(22) PCT Filed: Oct. 31, 2007
(86) PCT No.: PCT/US07/22967

§ 371 Date: Dec. 17, 2009

Related U.S. Application Data
(60) Provisional application No. 60/855,378, filed on Oct. 31, 2006.

(51) Int. Cl. G01N 33/53; C07K 16/00

(52) U.S. Cl. 435/7.92; 530/387.9

(57) ABSTRACT

Low molecular weight (LMW) peptides have been discovered that are indicative of breast cancer. Evaluating patient samples for the presence of such LMW peptides is an effective means of detecting breast cancer and monitoring the progression of the disease, for example during treatment. The LMW peptides are particularly useful in detecting breast cancer during its early stages.

### CROSS-REFERENCE TO RELATED PATENT APPLICATIONS

This application claims priority to U.S. Provisional Application No. 60/855,378, filed Oct. 31, 2006, which is hereby incorporated by reference.

### BACKGROUND

Breast cancer is the most frequent neoplasm and the leading cause of cancer mortality in women worldwide. According to estimates, approximately 41,000 women in the United States and 130,000 women in the European Union die for breast cancer yearly. Mammographic screening has been widespread for the past twenty years and shown to reduce breast cancer mortality by 20-35% in women aged 40 to 69 years.

Mammography has a variety of short-comings, however. For example, according to the current guidelines, some women who develop breast cancer are “too young” to start regular mammograms. Also, less than half of eligible women get mammograms regularly, and the predictive value of mammography declines in cohorts of patients with denser breast tissue and smaller lesions. Furthermore, mammography is not effective in detecting early stages of breast cancer.

Needless to say, early detection is of paramount importance in reducing mortality from this major public health burden. Detection of breast cancer at the earliest stages results in a much greater favorable outcome, with 10-year disease-free survival rate as high as 98% in patients with pT1a,bN0M0 tumors (measuring 1 cm or less, with disease-free axillary lymph nodes and no distant metastasis). Thus, the potential for enhancing treatment by providing an early diagnosis has driven a search for better diagnostic tools.

Some biomarker genes and proteins, such as BRCA1, BRCA2 and Her-2/neu, have been identified and developed into tools for genetic screening. The advantages and limitations of these detection approaches have been discussed in the literature. See, e.g. Ponzone et al., Eur. J. Cancer 34(7): 966-967, 1998; Bradbury, Lancet Oncol. 3: 2, 2002; Ross et al., Expert Rev. Mol. Diagn. 3(5): 573-585, 2003.

A need exists, however, for additional biomarkers useful for detecting breast cancer, and in particular biomarkers that can detect early stages of the disease.

### SUMMARY

In one embodiment, a method for detecting breast cancer in a patient comprises obtaining a biological sample from the patient and evaluating the sample or a fraction of the sample for the presence of at least one biomarker selected from the group of peptides having the sequence of SEQ ID NOs: 1-217, wherein the presence of said at least one biomarker is indicative of breast cancer. In one aspect, the methods involve evaluating the sample for the presence of a biomarker selected from the group of peptides having the amino acid sequence of SEQ ID NOs: 132-217. In another, the methods comprise evaluating the sample for the presence of peptides having the amino acid sequence of SEQ ID NOs: 132, 139, 141 and 148. In one aspect, the breast cancer is in early stage, such as stage T1a. The biological sample can be, for example, blood, serum or plasma. In one embodiment, the evaluation step comprises assays such as mass spectrometry, an immunoassay such as ELISA, immunomass spectrometry or suspension bead array.

In another embodiment, the method further comprises, prior to the evaluation step, harvesting low molecular weight peptides from the biological sample to generate at least one fraction comprising the peptides. In one embodiment, the size of the low molecular weight peptides is less than 50 KDa, preferably less than 25 KDa, and more preferably less than 15 KDa. In another aspect, the method also comprises digesting the low molecular weight peptides. Such digestion can be accomplished using enzymatic or chemical means. In one example, trypsin can be used to digest the peptides.

In another aspect, a method for monitoring the progression of breast cancer in a patient comprises (i) obtaining a biological sample from the patient, (ii) evaluating the sample or a fraction of the sample for the presence of at least one biomarker selected from the group of peptides having the sequences of SEQ ID NOs: 1-217, wherein the presence of said at least one biomarker is indicative of breast cancer, and optionally, repeating steps (i) and (ii) as necessary. In one aspect, the methods involve evaluating the sample for the presence of a biomarker selected from the group of peptides having the amino acid sequence of SEQ ID NOs: 132-217. In another, the methods comprise evaluating the sample for the presence of peptides having the amino acid sequence of SEQ ID NOs: 132, 139, 141 and 148. In one embodiment, the method further comprises a step of harvesting low molecular weight peptides from the sample to generate at least one fraction comprising the peptides.

In other aspects, the invention relates to antibodies specific for identified biomarkers for breast cancer, as well as kits for detecting breast cancer in a patient, comprising at least one such antibody.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 provides a CID Spectrum of peptide “CFVESLSSVETLK” from CDK4 protein identified only in LMW of breast cancer serum (accession number Q96BE9, amino acid residues 90 -102).

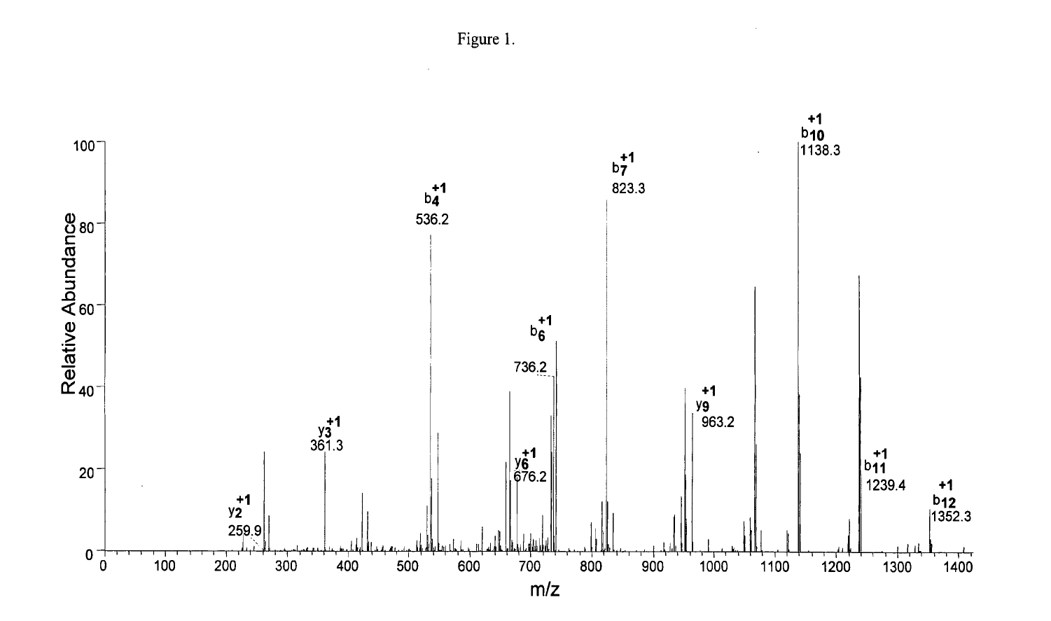

FIG. 2 provides a CID Spectrum of peptide “MVFHITTGSQEFDK” from meiotic recombination protein DMC1/LIM15 homolog identified only in LMW of breast cancer serum (accession number Q14565, amino acid residues 97-110).

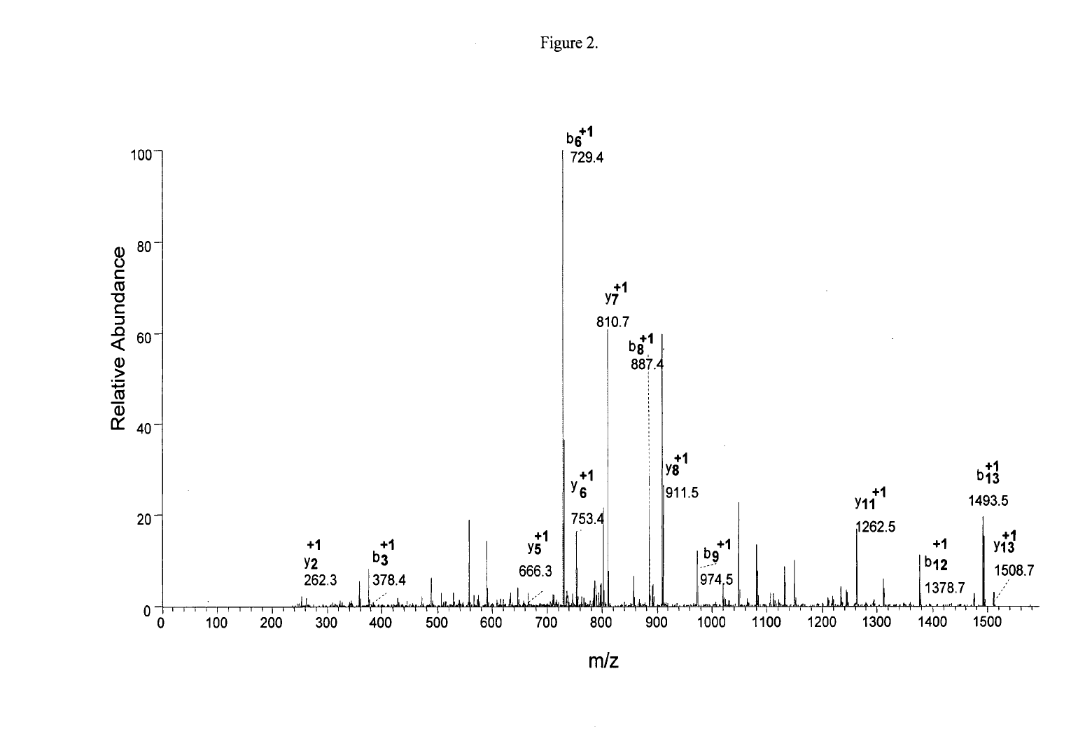

FIG. 3 provides a CID Spectrum of peptide “EVGNLLLENSQLLETK” from C-jun-amino-terminal kinase interacting protein 3 identified only in LMW of breast cancer serum (accession number Q9UPT6, amino acid residues 417-432).

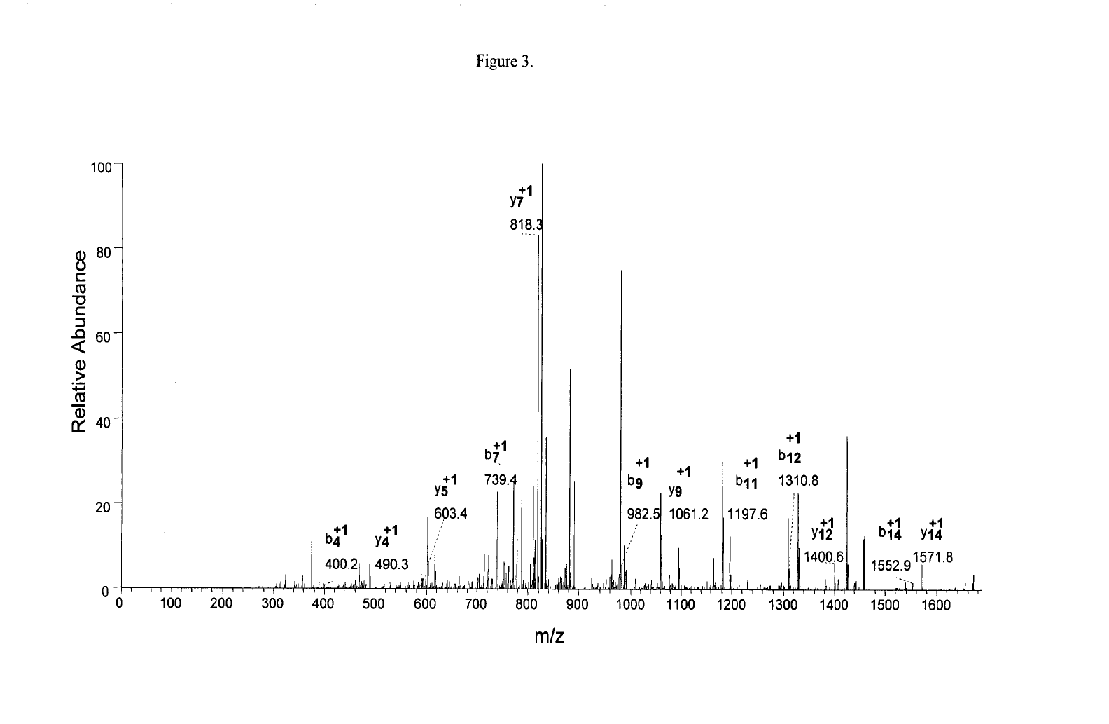

### DETAILED DESCRIPTION

Low molecular weight (LMW) peptides have been discovered that are indicative of breast cancer. Evaluating patient samples for the presence of such LMW peptides is an effective means of detecting breast cancer and monitoring the progression of the disease, for example during treatment. The LMW peptides are particularly useful in detecting breast cancer during its earliest stages, such as stage I.

The LMW peptides, or biomarkers, can be detected using a variety of methods known in the art. For example, antibodies can be utilized in immunoassays to detect the presence of a biomarker. Exemplary immunoassays include, e.g., ELISA, radioimmunoassay, immunofluorescent assay, “sandwich” immunoassay, western blot, immunoprecipitation assay and immunoelectrophoresis assays. Furthermore, methods involving beads, microbeads, arrays, microarrays, etc. can be applied in detecting the LMW peptides. Exemplary assays include, but are not limited to, suspension bead assays (Schwenk et al., “Determination of binding specificities in highly multiplexed bead-based assays for antibody proteomics,” Mol. Cell Proteomics, 6(1): 125-132 (2007)), antibody microarrays (Borrebaeck et al., “High-throughput proteomics using antibody microarrays: an update,” Expert Rev. Mol. Diagn. 7(5): 673-686 (2007)), aptamer arrays (Walter et al., “High-throughput protein arrays: prospects for molecular diagnostics,” Trends Mol. Med. 8(6): 250-253 (2002)), affybody arrays (Renberg et al., “Affibody molecules in protein capture microarrays: evaluation of multidomain ligands and different detection formats,” J. Proteome Res. 6(1): 171-179 (2007)), and reverse phase arrays (VanMeter et al., “Reverse-phase protein microarrays: application to biomarker discorvery and translational medicine,” Expert Rev. Mol. Diagn. 7(5): 625-633 (2007)). All these publications are incorporated herein by reference.

In another example, the inventive biomarkers can be detected using mass spectrometry (MS). One example of this approach is tandem mass spectrometry (MS/MS), which involves multiple steps of mass selection or analysis, usually separated by some form of fragmentation. Most such assays use electrospray ionization followed by two stages of mass selection: a first stage (MS 1) selecting the mass of the intact analyte (parent ion) and, after fragmentation of the parent by collision with gas atoms, a second stage (MS2) selecting a specific fragment of the parent, collectively generating a selected reaction monitoring assay. In one embodiment, collision-induced dissociation is used to generate a set of fragments from a specific peptide ion. The fragmentation process primarily gives rise to cleavage products that break along peptide bonds. Because of the simplicity in fragmentation, the observed fragment masses can be compared to a database of predicted masses for known peptide sequences. A number of different algorithmic approaches have been described to identify peptides and proteins from tandem mass spectrometry (MS/MS) data, including peptide fragment fingerprinting (SEQUEST, MASCOT, OMSSA and X!Tandem), peptide de novo sequencing (PEAKS, LuteFisk and Sherenga) and sequence tag based searching (SPIDER, GutenTAG).

10018] Likewise, multiple reaction monitoring (MRM) can be used to identify the inventive biomarkers in patient samples. This technique applies the MS/MS approach to, for example, tryptic digests of the input sample, followed by selected ion partitioning and sampling using MS to objectify and discreetize the analyte if interest by following the exact m/z ion of the tryptic fragment that represents the analyte. Such an approach can be performed in multiplex so that multiple ions can be measured at once, providing an antibody-free method for analyte measurement. See, e.g. Andersen et al., “Quantitative mass spectrometric multiple reaction monitoring assays for major plasma proteins,” Molecular & Cellular Proteomics, 5.4: 573-588 (2006); Whiteaker et al., “Integrated pipeline for mass spectrometry-based discorvery and confirmation of biomarkers demonstrated in a mouse model of breast cancer,” J. Proteome Res. 6(10): 3962-75 (2007). Both publications are incorporated herein by reference in their entirety.

In another example, the inventive biomarkers can be detected using nanoflow reverse-phase liquid chromatography-tandem mass spectrometry. See, e.g., Domon B, Aebersold R. “Mass spectrometry and protein analysis.” Science, 312(5771):212-7(2006), which is incorporated herein by reference in its entirety. Using this approach, experimentalists obtain peptide fragments, usually by trypsin digest, and generate mass spectrograms of the fragments, which are then compared to a database, such as SEQUEST, for protein identification.

In another aspect, the inventive biomarkers can be detected using immuno-mass spectrometry. See, e.g., Liotta L et al. “Serum peptidome for cancer detection: spinning biologic trash into diagnostic gold.” J Clin Invest.,116(1):26-30 (2006); Nedelkov, “Mass spectrometry-based immunoassays for the next phase of clinical applications,” Expert Rev. Proteomics, 3(6): 631-640 (2006), which are incorporated herein by reference. Immuno-mass spectrometry provides a means for rapidly determining the exact size and identity of a peptide biomarker isoform present within a patient sample. When developed as a high throughput diagnostic assay, a drop of patient's blood, serum or plasma can be applied to a high density matrix of microcolumns or microwells filled with a composite substratum containing immobilized polyclonal antibodies, directed against the peptide marker. All isoforms of the peptide that contain the epitope are captured. The captured population of analytes including the analyte fragments are eluted and analyzed directly by a mass spectrometer such as MALDI-TOF MS. The presence of the specific peptide biomarker at its exact mass/charge (m/z) location would be used as a diagnostic test result. The analysis can be performed rapidly by simple software that determines if a series of ion peaks are present at defined m/z locations.

In yet another example, the inventive biomarkers can be detected using standard immunoassay-based approaches whereby fragment specific antibodies are used to measure and record the presence of the diagnostic fragments. See, e.g., Naya et al. “Evaluation of precursor prostate-specific antigen isoform ratios in the detection of prostate cancer.” Urol Oncol. 23(1):16-21 (2005). Moreover, additional immunoassays are well known to those skilled in the field, such as ELISAs (Maeda et al., “Blood tests for asbestos-related mesothelioma,” Oncology 71: 26-31 (2006)), microfluidic ELISA (Lee et al., “Microfluidic enzyme-linked immunosorbent assay technology,” Adv. Clin. Chem. 42: 255-259 (2006)), nanocantilever immunoassays (Kurosawa et al., “Quartz crystal microbalance immunosensors for environmental monitoring,” Biosens Bioelectron, 22(4): 473-481 (2006)), and plasmon resonance immunoassays (Nedelkov, “Development of surface Plasmon resonance mass spectrometry array platform,” Anal. Chem. 79(15): 5987-5990 (2007)). All of these publications are incorporated herein by reference.

In a further example, the biomarkers can be detected using electrochemical approaches. See, e.g., Lin et al., “Electrochemical immunosensor for carcinoembryonic antigen based on antigen immobilization in gold nanoparticles modified chitosan membrane,” Anal. Sci. 23(9): 1059-1063 (2007).

In one embodiment, the LMW peptides are harvested from a biological sample prior to the evaluation step. For example, 100 μl of serum can be mixed with 2× SDS-PAGE Laemmli Buffer (containing 200 mM DTT), boiled for 10 minutes, and loaded on Prep Cell (Model 491 Prep Cell, Bio-Rad Laboratories, CA) comprising a 5 cm length 10% acrylamide gel. Electrophoresis is performed under a constant voltage of 250V. Immediately after the bromophenol blue indicator dye is eluted from the system, LMW peptides and proteins migrate out of the gel and are trapped in a dialysis membrane in the elution chamber. These molecules can be eluted at a flow rate of 400 ml/l min by a buffer with the same composition of the Tris-Glycine running buffer and collected for 10 minutes in one fraction.

Alternatively, LMW peptides can be harvested using from a sample using a capture-particle that comprises a molecular sieve portion and an analyte binding portion as described in U.S. patent application Ser. No. 11/527,727, filed Sep. 27, 2006, which is incorporated herein by reference in its entirety. Briefly, either the molecular sieve portion or the analyte binding portion or both comprise a cross-linked region having modified porosity, or pore dimensions sufficient to exclude high molecular weight molecules.

In another embodiment, the LMW peptides are digested prior to detection, so as to reduce the size of the peptides. Such digestion can be carried out using standard methods well known in the field. Exemplary treatments, include but are not limited to, enzymatic and chemical treatments. Such treatments can yield partial as well as complete digestions. One example of an enzymatic treatment is a trypsin digestion.

The inventive biomarkers are particularly useful in detecting breast cancer during its early stages, i.e., prior to metastasis and large tumor volume (e.g. greater than 2 cm).

Antibodies specific for the inventive biomarkers can be produced readily using well known methods in the art. (See, J. Sambrook, E. F. Fritsch and T. Maniatis, Molecular Cloning, a Laboratory Manual, second edition, Cold Spring Harbor Laboratory Press, pp. 18.7-18.18, 1989) For example, the inventive biomarkers can be prepared readily using an automated peptide synthesizer. Next, injection of an immunogen, such as (peptide)ₙ-KLH (n=1-30) in complete Freund's adjuvant, followed by two subsequent injections of the same immunogen suspended in incomplete Freund's adjuvant into immunocompetent animals, is followed three days after an i.v. boost of antigen, by spleen cell harvesting. Harvested spleen cells are then fused with Sp2/0-Ag14 myeloma cells and culture supernatants of the resulting clones analyzed for anti-peptide reactivity using a direct-binding ELISA. Fine specificity of generated antibodies can be detected by using peptide fragments of the original immunogen.

In certain embodiments, one or more antibodies directed to the inventive biomarkers is provided in a kit, for use in a diagnostic method. Such kits also can comprise reagents, instructions and other products for performing the diagnostic method.

The detailed description of the present invention is provided below by the following example, which is illustrative only and not limiting the invention in any way.

### Examples

### Example 1

### Identification of Biomarkers for Breast Cancer Using LTQ

Blood Collection and Serum Preparation

Blood samples were drawn from patients before the mammography screening under full Institutional Review Board approval and patient's consent. Specimens were collected in red-top Vacutainer Tubes and allowed to clot for 1 hour on ice, followed by centrifugation at 4° C. for 10 minutes at 2000 g. The serum supernatant was divided in aliquots and stored at −80° C. until needed. 10 serum samples with negative outcome were pooled in a single control group. 10 serum samples from patients with a diagnosed T1a stage breast cancer were pooled in a single disease group. Each experiment has been performed using 3 different aliquots from the same pool, both, for the control and for the disease group.

Low Molecular Weight (LMW) Protein Harvesting by Continuous Elution Electrophoresis

100 μl of serum was mixed with 2× SDS-PAGE Laemmli Buffer (containing 200 mM DTT), boiled for 10 minutes, and loaded on Prep Cell (Model 491 Prep Cell, Bio-Rad Laboratories, CA) in which 5 cm length 10% acrylamide gel was polymerized. Electrophoresis was performed under a constant voltage of 250V. Immediately after the bromophenol blue indicator dye was eluted from the system, LMW peptides and proteins migrate out of the gel and are trapped in a dialysis membrane in the elution chamber. These molecules were eluted at a flow rate of 400 μl min by a buffer with the same composition of the Tris-Glycine running buffer and collected for 10 minutes in one fraction.

SDS Removal from the Prep Cell Fractions

LMW fractions obtained by the Prep Cell were processed using a commercially available ion-exchange matrix (Proteo Spin Detergent Clean-Up Micro Kit, Norgen Biotek Corporation, Canada) following protocols outlined by the manufacturer for both acidic and basic proteins, resulting in a final volume of 55 μl.

Nanoflow Reversed-Phase Liquid Chromatography-Tandem MS (nanoRPLC-MS/MS)

The SDS-free LMW fractions obtained from the described procedure were analyzed by traditional bottom-up MS approaches. This was accomplished by treating the samples by reduction using 20 mM DTT, followed by alkylation using 100 mM iodoacetamide and lastly, trypsin digestion (Promega, WI) at 37° C. overnight in 50 mM ammonium bicarbonate in the presence of 1M urea in a final volume of 200 pl. Tryptic peptides were desalted by μC₁₈ Zip Tip (Millipore, MA) and analyzed by reversed-phase liquid chromatography nanospray tandem mass spectrometry using a linear ion-trap mass spectrometer (LTQ, ThermoElectron, San Jose, Calif.). Reverse phase column was slurry-packed in-house with 5 μm, 200 Å pore size C₁₈ resin (Michrom BioResources, CA) in 100 μm i.d.×10 cm long fused silica capillary (Polymicro Technologies, Phoenix, Ariz.) with a laser-pulled tip. After sample injection, the column was washed for 5 min with mobile phase A (0.4% acetic acid, 0.005% heptafluorobutyric acid) and peptides were eluted using a linear gradient of 0% mobile phase B (0.4% acetic acid, 0.005% heptafluorobutyric acid, 80% acetonitrile) to 50% mobile phase B in 30 min at 250 nl/min, then to 100% B in an additional 5 min. The LTQ mass spectrometer was operated in a data-dependent mode in which each full MS scan was followed by five MS/MS scans where the five most abundant molecular ions were dynamically selected and fragmented by collision-induced dissociation (CID) using a normalized collision energy of 35%.

Bioinformatic Analysis

Tandem mass spectra were matched against Swiss-Prot human protein database through SEQUEST algorithm incorporated in Bioworks software (version 3.2, Thermo Electron) using tryptic cleavage constraints and static cysteine alkylation by iodoacetamide. For a peptide to be considered legitimately identified, it had to achieve Delta Cn value above 0.1, cross correlation scores of 1.5 for [M+H]¹⁺, 2.0 for [M+2H]²⁺, 2.5 for [M+3H]³⁺, and a probability cut-off for randomized identification of p<0.01.

The results are provided in Table 1. In short, 131 peptides were identified as biomarkers that correlate to the disease state. Thus, evaluating patient samples for the presence of one or more of these biomarkers will provide a useful method for detecting breast cancer.

TABLE 1

| | | | | | | |
|---|---|---|---|---|---|---|
| | | | | | SEQ | |
| | | | | | ID | Residue |
| Proteins | P (pro) | MW | Accession | Amino acid sequence | NO. | number |

| | | | | | | |
|---|---|---|---|---|---|---|
| NOTC1_HUMAN (P46531) Neurogenic | 2.96E−03 | 272372.2 | P46531 | QWTQQHLDAADLR | 1 | 1843-1855 |
| locus notch homolog protein | | | | | | |
| 1 precursor | | | | | | |
| (Notch 1) (hN1) (Translocat | | | | | | |
| Q4G171 (Q4G171) CASC1 protein | 4.95E−03 | 79322.33 | Q4G171 | QASTLADLDSGNMEK | 2 | 254-268 |
| Q69YN4 (Q69YN4) Hypothetical protein | 4.77E−04 | 201574 | Q69YN4 | NLRFEINCIPNLIEYVK | 3 | 898-914 |
| DKFZp686C1522 | | | | | | |
| Q8N3Y7 (Q8N3Y7) Epidermal retinal | 6.86E−03 | 34072.78 | Q8N3Y7 | KEVGDVSILINNAGIVTGK | 4 | 114-132 |
| dehydrogenase 2 | | | | | | |
| PSD1_HUMAN (Q99460) 26S proteasome | 4.79E−03 | 105768.7 | Q99460 | NNNTDLMILKNTKDAVR | 5 | 345-361 |
| non-ATPase regulatory subunit 1 (26S | | | | | | |
| proteasome regulatory subun | | | | | | |
| KCNH6_HUMAN (Q9H252) Potassium | 7.88E−04 | 109855.1 | Q9H252 | GSIEILRDDVVVAILGK | 6 | 637-653 |
| voltage-gated channel subfamily H | | | | | | |
| member 6 (Voltage-gated potassium | | | | | | |
| WDR36_HUMAN (Q8NI36) WD-repeat | 6.93E−03 | 105255.2 | Q8NI36 | TASALFAGF | 7 | 65-88 |
| protein 36 (T-cell activation WD repeat | | | | RALGLFSNDIPHVVR | | |
| protein) (TA-WDRP) | | | | | | |
| FLRT2_HUMAN (O43155) Leucine-rich | 1.92E−03 | 74001.84 | O43155 | ERVTPPISER | 8 | 415-424 |
| repeat transmembrane protein FLRT2 | | | | | | |
| precursor (Fibronectin-like do | | | | | | |
| PCNT2_HUMAN (O95613) Pericentrin-2 | 1.16E−03 | 377847.9 | O95613 | AAGSDADHLREQQR | 9 | 2960-2973 |
| (Pericentrin B) (Kendrin) | | | | | | |
| Q15813 (Q15813) Beta-tubulin cofactor E | 5.90E−05 | 59309.08 | Q15813 | PNKVNFGTDFLTAIKNR | 10 | 72-88 |
| (Tubulin-specific chaperone e) | | | | | | |
| CAN12_HUMAN (Q6ZSI9) Calpain-12 (EC | 4.58E−03 | 80985.57 | Q6ZSI9 | TPKCTVLLSLIQR | 11 | 421-433 |
| 3.4.22.—) | | | | | | |
| Q96BB9 (Q96BB9) IGHM protein | 1.14E−03 | 64998.24 | Q96BB9 | DTLYLQMNSLR | 12 | 96-106 |
| SNX23_HUMAN (Q96L93) Kinesin-like | 4.35E−04 | 151916.8 | Q96L93 | TLKLKYAELAALEFPPK | 13 | 1230-1246 |
| motor protein C20orf23 (Sorting | | | | | | |
| nexin 23) | | | | | | |
| E41LB_HUMAN (Q9H329) Band 4.1-like | 9.77E−03 | 100657.1 | Q9H329 | SPAQAELSYLNKAK | 14 | 240-253 |
| protein 4B (EHM2 protein) (FERM- | | | | | | |
| containing protein CG1) | | | | | | |
| EPHA5_HUMAN (P54756) Ephrin type-A | 4.48E−03 | 114710.3 | P54756 | RLGVTLVGHQK | 15 | 1007-1017 |
| receptor 5 precursor (EC 2.7.1.112) | | | | | | |
| (Tyrosine-protein kinase rec | | | | | | |
| PSA7L_HUMAN (Q8TAA3) Proteasome | 2.43E−03 | 28540.14 | Q8TAA3 | EVELYVTEIEKEKEEAEK | 16 | 232-249 |
| subunit alpha type 7-like (EC 3.4.25.1) | | | | | | |
| Q9BVV2 (Q9BVV2) Hypothetical protein | 5.03E−03 | 36503.15 | Q9BVV2 | DSLTLHTKPEPLEG | 17 | 298-318 |
| MGC5356 (OTTHUMP00000031567) | | | | PALSHSV | | |
| MRGBP_HUMAN (Q9NV56) MRG-binding | 4.77E−03 | 22402.99 | Q9NV56 | EDVDPHNGADDVFSS | 18 | 132-156 |
| protein | | | | SGSLGKASEK | | |
| JIP3_HUMAN (Q9UPT6) C-jun-amino- | 3.33E−03 | 146961.6 | Q9UPT6 | EVGNLLLENSQLLETK | 19 | 417-432 |
| terminal kinase interacting protein | | | | | | |
| 3 (JNK-interacting protein 3) ( | | | | | | |
| LG3BP_HUMAN (Q08380) Galectin-3 | 2.47E−03 | 65289.4 | Q08380 | SDLAVPSELALLK | 20 | 311-323 |
| binding protein precursor (Lectin | | | | | | |
| galactoside-binding soluble 3 bin | | | | | | |
| SNX17_HUMAN (Q15036) Sorting nexin- | 1.58E−03 | 52868.24 | Q15036 | SPPLLESPDATRESMVKLSSK | 21 | 415-435 |
| 17 | | | | | | |
| Q8N8J8 (Q8N8J8) Hypothetical protein | 8.60E−03 | 35709.23 | Q8N8J8 | EACIVEALGIQTLTNQK | 22 | 257-273 |
| FLJ39369 | | | | | | |
| Q9NU63 (Q9NU63) Chromosome 6 open | 6.64E−04 | 51869.71 | Q9NU63 | MAAGEPRSLLFFQK | 23 | 1-14 |
| reading frame 40 (Fragment) | | | | | | |
| DMC1_HUMAN (Q14565) Meiotic | 3.02E−03 | 37657.33 | Q14565 | MVFHITTGSQEFDK | 24 | 97-110 |
| recombination protein DMC1/LIM15 | | | | | | |
| homolog | | | | | | |
| THAP4_HUMAN (Q8WY91) THAP domain | 8.64E−03 | 62850.72 | Q8WY91 | FIGSLHSYSFSSKHTR | 25 | 231-246 |
| protein 4 | | | | | | |
| ROBO4_HUMAN (Q8WZ75) Roundabout | 1.83E−04 | 107390.4 | Q8WZ75 | PAVWLSWK | 26 | 262-285 |
| homolog 4 precursor (Magic roundabout) | | | | VSGPAAPAQSYTALFR | | |
| HTRA1_HUMAN (Q92743) Serine | 6.04E−03 | 51254.68 | Q92743 | YIGIRMMSLTSSK | 27 | 382-394 |
| protease HTRA1 precursor (EC 3.4.21.—) | | | | | | |
| (L56) | | | | | | |
| TLR8_HUMAN (Q9NR97) Toll-like receptor | 8.10E−03 | 119752.5 | Q9NR97 | NLYLAWNCYFNKVCEK | 28 | 174-189 |
| 8 precursor | | | | | | |
| HXA3_HUMAN (O43365) Homeobox | 5.13E−03 | 46339.7 | O43365 | VEMANLLNLTERQIK | 29 | 222-236 |
| protein Hox-A3 (Hox-1E) | | | | | | |
| Q6ZR27 (Q6ZR27) Hypothetical protein | 6.97E−03 | 54430.06 | Q6ZR27 | LVEVIPEGAMLRLG | 30 | 208-237 |
| FLJ46705 | | | | MTNPPYILEHLEEMAK | | |
| Q6ZRS3 (Q6ZRS3) Hypothetical protein | 6.45E−04 | 17827.01 | Q6ZRS3 | PAASPNTTSSRGQTV | 31 | 23-46 |
| FLJ46148 | | | | HPPCSSKLR | | |
| Q8TAM1 (Q8TAM1) Hypothetical protein | 4.40E−03 | 73940.78 | Q8TAM1 | NRLTDYYEPLLKN | 32 | 465-485 |
| FLJ23560 | | | | NSTAYSTR | | |
| BCL9_HUMAN (O00512) B-cell lymphoma | 9.09E−03 | 149218.7 | O00512 | FAMPSSTPLYHDAIK | 33 | 1016-1030 |
| 9 protein (Bcl-9) (Legless homolog) | | | | | | |
| AKAP3_HUMAN (O75969) A-kinase | 9.40E−05 | 94676.41 | O75969 | SCDASLAELGDDKSGDASR | 34 | 687-705 |
| anchor protein 3 (Protein kinase A | | | | | | |
| anchoring protein 3) (PRKA3) (A-ki | | | | | | |
| O95973 (O95973) VH4 heavy chain | 1.29E−05 | 16305.14 | O95973 | VTISVDTSK | 35 | 88-96 |
| variable region precursor (Fragment) | | | | | | |
| Q5SZH6 (Q5SZH6) Novel protein | 6.74E−04 | 53734.77 | Q5SZH6 | MDINTYNNQLHLQR | 36 | 1-14 |
| Q9UL71 (Q9UL71) Myosin-reactive | 9.27E−03 | 13145.42 | Q9UL71 | AEDTALYYCAK | 37 | 88-98 |
| immunoglobulin heavy chain variable | | | | | | |
| region (Fragment) | | | | | | |
| MFA3L_HUMAN (O75121) Microfibrillar- | 1.22E−03 | 45351.02 | O75121 | SHLTVCFLPSVPF | 38 | 6-29 |
| associated protein 3-like precursor | | | | LILVSTLATAK | | |
| (Protein kinase NYD-SP9) | | | | | | |
| GOGA4_HUMAN (Q13439) Golgi | 7.07E−04 | 260978.6 | Q13439 | ELSENINAVTLMKEELKEK | 39 | 1347-1365 |
| autoantigen, golgin subfamily A | | | | | | |
| member 4 (Trans-Golgi p230) | | | | | | |
| (256 kDa gol | | | | | | |
| AUTS2_HUMAN (Q8WXX7) Autism | 4.77E−03 | 138897.1 | Q8WXX7 | PGQNSCRDSDSES | 40 | 177-199 |
| susceptibility gene 2 protein | | | | ASGESKGFHR | | |
| CELR1_HUMAN (Q9NYQ6) Cadherin EGF | 6.96E−03 | 329276.7 | Q9NYQ6 | DANSVITYQLTGGNTR | 41 | 715-730 |
| LAG seven-pass G-type receptor 1 | | | | | | |
| precursor (Flamingo homolog 2) ( | | | | | | |
| WRN_HUMAN (Q14191) Werner | 1.41E−03 | 162390.4 | Q14191 | DEIQCVIATIAFGMGINKADIR | 42 | 813-834 |
| syndrome helicase | | | | | | |
| Q5R329 (Q5R329) Testicular soluble | 9.72E−03 | 187025.2 | Q5R329 | AVIKNRNTTYIV | 43 | 685-708 |
| adenylyl cyclase (SAC) | | | | IGAVQPNDISNK | | |
| Q5TF21 (Q5TF21) | 1.66E−03 | 103136.2 | Q5TF21 | VMQLQYENRVLMSNMQRY | 44 | 719-745 |
| OTTHUMP00000017175 | | | | DLASHLGIR | | |
| Q9H0R6 (Q9H0R6) Hypothetical protein | 3.65E−03 | 57432.23 | Q9H0R6 | GRILSGNFFLLKENYENYFVK | 45 | 375-395 |
| DKFZp564C1278 | | | | | | |
| TENX_HUMAN (P22105) Tenascin-X | 8.10E−03 | 464165.9 | P22105 | DRDGRPQVVR | 46 | 2203-2212 |
| precursor (TN-X) (Hexabrachion-like | | | | | | |
| protein) | | | | | | |
| DOCK1_HUMAN (Q14185) Dedicator of | 2.23E−03 | 215237.7 | Q14185 | NVEVTVSVYDEDGKR | 47 | 447-461 |
| cytokinesis protein 1 (180 kDa protein | | | | | | |
| downstream of CRK) (DOCK18 | | | | | | |
| DKK4_HUMAN (Q9UBT3) Dickkopf-related | 6.46E−03 | 24859.18 | Q9UBT3 | KGQEGESC | 48 | 138-159 |
| protein 4 precursor (Dkk-4) | | | | LRTFDCGPGLCCAR | | |
| (Dickkopf-4) (hDkk-4) | | | | | | |
| [Contains: D | | | | | | |
| RPC1_HUMAN (O14802) DNA-directed | 3.06E−03 | 155648.3 | O14802 | TCCHIMLSQEEK | 49 | 111-122 |
| RNA polymerase III largest subunit (EC | | | | | | |
| 2.7.7.6) (RPC155) (RPC1) | | | | | | |
| FA10_HUMAN (P00742) Coagulation | 8.74E−03 | 54696.55 | P00742 | CKDGLGEYTCTCLEGFEGK | 50 | 101-119 |
| factor X precursor (EC 3.4.21.6) | | | | | | |
| (Stuart factor) (Stuart-Prower fac | | | | | | |
| XCL1_HUMAN (P47992) Lymphotactin | 3.14E−03 | 12508.7 | P47992 | AVIFITKRGLK | 51 | 57-67 |
| precursor (XCL1) (Cytokine SCM-1) | | | | | | |
| (ATAC) (Lymphotaxin) (SCM-1-alph | | | | | | |
| Q4J6C4 (Q4J6C4) Prolyl endopeptidase- | 3.44E−03 | 76692.8 | Q4J6C4 | QENEKPLPENMDAFEKVR | 52 | 80-97 |
| like variant E | | | | | | |
| Q6P387 (Q6P387) C16orf46 protein | 1.20E−03 | 42723.96 | Q6P387 | AKEFIIGTGWEEAVQGWGR | 53 | 58-76 |
| CI068_HUMAN (Q8N4H0) Protein C9orf68 | 2.80E−04 | 45029.9 | Q8N4H0 | LPKGMQARAPSQYSTR | 54 | 178-193 |
| ANKR6_HUMAN (Q9Y2G4) Ankyrin repeat | 7.67E−03 | 75675.76 | Q9Y2G4 | EEAREEFLSASPEPR | 55 | 283-297 |
| domain protein 6 | | | | | | |
| DKC1_HUMAN (O60832) H/ACA | 2.91E−03 | 57506.79 | O60832 | LDTSQWPLLLK | 56 | 46-56 |
| ribonucleoprotein complex subunit 4 (EC | | | | | | |
| 5.4.99.—) (Dyskerin) (Nucleolar p | | | | | | |
| FIBB_HUMAN (P02675) Fibrinogen beta | 4.12E−08 | 55892.23 | P02675 | EEAPSLRPAPPPISGGGYR | 57 | 54-72 |
| chain precursor [Contains: | | | | | | |
| Fibrinopeptide B] | | | | | | |
| K1C18_HUMAN (P05783) Keratin, type I | 5.18E−05 | 47897.57 | P05783 | DWSHYFKIIEDLRA | 58 | 124-148 |
| cytoskeletal 18 (Cytokeratin-18) | | | | QIFANTVDNAR | | |
| (CK-18) (Keratin-18) (K18) | | | | | | |
| Q6P1M6 (Q6P1M6) Insulin-like growth | 6.24E−03 | 31653.76 | Q6P1M6 | FLNVLSPR | 59 | 226-233 |
| factor binding protein 3 | | | | | | |
| WDR9_HUMAN (Q9NSI6) WD-repeat | 5.21E−03 | 257060.4 | Q9NSI6 | QNCKGDSQPNK | 60 | 1442-1452 |
| protein 9 | | | | | | |
| NCOR2_HUMAN (Q9Y618) Nuclear | 7.54E−04 | 273863.1 | Q9Y618 | VVTLAQHISEVITQDYTR | 61 | 2132-2149 |
| receptor corepressor 2 (N-CoR2) | | | | | | |
| (Silencing mediator of retinoic acid a | | | | | | |
| ACTN1_HUMAN (P12814) Alpha-actinin 1 | 2.98E−03 | 102992.7 | P12814 | ICDQWDNLGALTQKRR | 62 | 479-494 |
| (Alpha-actinin cytoskeletal isoform) | | | | | | |
| (Non-muscle alpha-actinin | | | | | | |
| LAMB3_HUMAN (Q13751) Laminin beta-3 | 7.23E−03 | 129488.5 | Q13751 | LGQSSMLGEQGARIQSVK | 63 | 1092-1109 |
| chain precursor (Laminin 5 beta 3) | | | | | | |
| (Laminin B1k chain) (Kalinin | | | | | | |
| RYR3_HUMAN (Q15413) Ryanodine | 7.53E−03 | 551577.3 | Q15413 | SCQSGEDEEEDEDKEKTFEEK | 64 | 3587-3607 |
| receptor 3 (Brain-type ryanodine | | | | | | |
| receptor) (RyR3) (RYR-3) | | | | | | |
| (Brain ryan | | | | | | |
| MK06_HUMAN (Q16659) Mitogen- | 6.39E−03 | 82628.7 | Q16659 | ALSDVTDEEEVQVDPRK | 65 | 384-400 |
| activated protein kinase 6 | | | | | | |
| (EC 2.7.1.37) (Extracellular | | | | | | |
| signal-regulate | | | | | | |
| OR5DI_HUMAN (Q8NGL1) Olfactory | 4.67E−03 | 35324.45 | Q8NGL1 | DVKDTVTEILDTKVFSY | 66 | 297-313 |
| receptor 5D18 | | | | | | |
| Q96BE9 (Q96BE9) CDK4 protein | 4.81E−03 | 11993.24 | Q96BE9 | CFVESLSSVETLK | 67 | 90-102 |
| Q9C063 (Q9C063) LYST-interacting | 3.34E−03 | 25900.14 | Q9C063 | PCWELKKIMILK | 68 | 167-178 |
| protein LIP5 (Fragment) | | | | | | |
| Q9HBL8 (Q9HBL8) HSCARG | 6.57E−04 | 33323.32 | Q9HBL8 | DIGVPMTSVRLPCYF | 69 | 142-167 |
| | | | | ENLLSHFLPQK | | |
| RRBP1_HUMAN (Q9P2E9) Ribosome- | 1.65E−03 | 152380 | Q9P2E9 | ADSVANQGTKVEGITNQGKK | 70 | 550-569 |
| binding protein 1 (Ribosome receptor | | | | | | |
| protein) (180 kDa ribosome recep | | | | | | |
| HV3H_HUMAN (P01769) Ig heavy chain | 3.23E−06 | 13157.39 | P01769 | AENTAVYYCAR | 71 | 88-98 |
| V-III region GA | | | | | | |
| SEM3F_HUMAN (Q13275) Semaphorin-3F | 1.27E−03 | 88325.28 | Q13275 | EPLIIHWAAS | 72 | 88-109 |
| precursor (Semaphorin IV) (Sema IV) | | | | PQRIEECVLSGK | | |
| (Sema III/F) | | | | | | |
| TXND2_HUMAN (Q86VQ3) Thioredoxin | 1.47E−03 | 60424.09 | Q86VQ3 | MDVDKELGMESVK | 73 | 1-13 |
| domain-containing protein 2 (Spermatid- | | | | | | |
| specific thioredoxin-1) (Sp | | | | | | |
| Q8N9P0 (Q8N9P0) Hypothetical protein | 3.11E−03 | 25142.83 | Q8N9P0 | DVAGARGAPPAWGQAPSPRR | 74 | 178-197 |
| FLJ36797 | | | | | | |
| Q96CS4 (Q96CS4) Zinc finger protein | 4.07E−03 | 56870.35 | Q96CS4 | ETYGHLGALGCAGPK | 75 | 60-74 |
| HIT-39 (Hypothetical protein | | | | | | |
| FLJ90415) | | | | | | |
| Q96MT7 (Q96MT7) Hypothetical protein | 2.25E−03 | 111658.3 | Q96MT7 | KKILDADIQLK | 76 | 558-568 |
| FLJ31910 | | | | | | |
| Q96PE2 (Q96PE2) Tumor endothelial | 6.84E−04 | 221533.1 | Q96PE2 | PKMLVISGGDGYED | 77 | 2022-2051 |
| marker 4 | | | | TVGRFRLSSGGGSSSE | | |
| ICAM5_HUMAN (Q9UMF0) Intercellular | 7.47E−03 | 97270.1 | Q9UMF0 | EPETQPVCFFR | 78 | 92-102 |
| adhesion molecule 5 precursor (ICAM-5) | | | | | | |
| (Telencephalin) | | | | | | |
| AP4B1_HUMAN (Q9Y6B7) Adapter-related | 9.85E−03 | 83207.77 | Q9Y6B7 | GPLLAACSSES | 79 | 279-301 |
| protein complex 4 beta 1 subunit (Beta | | | | RELCFVALCHVR | | |
| subunit of AP-4) (AP-4 | | | | | | |
| CBPD_HUMAN (O75976) | 3.19E−03 | 152818.8 | O75976 | GASSSTNDASVPTTKEFETLIK | 80 | 886-907 |
| Carboxypeptidase D precursor (EC | | | | | | |
| 3.4.17.22) (Metallocarboxypeptidase D) | | | | | | |
| (gp180) | | | | | | |
| TRY1_HUMAN (P07477) Trypsin I | 2.00E−03 | 26541.09 | P07477 | NKPGVYTKVYNYVK | 81 | 224-237 |
| precursor (EC 3.4.21.4) (Cationic | | | | | | |
| trypsinogen) | | | | | | |
| S61A1_HUMAN (P61619) Protein | 4.32E−04 | 52099.53 | P61619 | GTLMELGISPIVT | 82 | 73-97 |
| transport protein Sec61 alpha subunit | | | | SGLIMQLLAGAK | | |
| isoform 1 (Sec61 alpha-1) | | | | | | |
| AT8B2_HUMAN (P98198) Probable | 5.19E−03 | 137351.9 | P98198 | ENKFPLSNQNMLLR | 83 | 238-251 |
| phospholipid-transporting ATPase ID (EC | | | | | | |
| 3.6.3.1) (ATPase class I type | | | | | | |
| Q6PIZ8 (Q6PIZ8) TRAV20 protein | 2.83E−03 | 30530.23 | Q6PIZ8 | GRGSQGNLIFGKGTK | 84 | 113-127 |
| Q96MY8 (Q96MY8) Hypothetical protein | 2.56E−03 | 55323.95 | Q96MY8 | ISSTSTDR | 85 | 177-184 |
| FLJ31695 | | | | | | |
| TRIM8_HUMAN (Q9BZR9) Tripartite motif | 5.83E−03 | 61449.34 | Q9BZR9 | QTVEVLDK | 86 | 249-256 |
| protein 8 (RING finger protein 27) | | | | | | |
| (Glioblastoma-expressed RI | | | | | | |
| Q9UCY0 (Q9UCY0) OVCA1 = CANDIDATE | 9.18E−03 | 48820.3 | Q9UCY0 | QVMAALVVSGAAEQGGR | 87 | 4-20 |
| tumor suppressor | | | | | | |
| AHNK_HUMAN (Q09666) Neuroblast | 5.40E−05 | 312292.7 | Q09666 | GEIDASVPELEG | 88 | 1550-1571 |
| differentiation-associated protein | | | | DLRGPQVDVK | | |
| AHNAK (Desmoyokin) (Fragments) | | | | | | |
| TTLL7_HUMAN (Q6ZT98) Tubulin tyrosine | 1.22E−03 | 102933.8 | Q6ZT98 | YLLPGSTQFFLRTPTYNLK | 89 | 849-867 |
| ligase-like protein 7 (Protein | | | | | | |
| NYD-SP30) | | | | | | |
| SYCP2_HUMAN (Q9BX26) Synaptonemal | 8.81E−03 | 175528.1 | Q9BX26 | ESKKLLTIILK | 90 | 347-357 |
| complex protein 2 (SCP-2 protein) | | | | | | |
| (Synaptonemal complex lateral e | | | | | | |
| DDEF2_HUMAN (O43150) Development | 4.59E−03 | 111580.7 | O43150 | EIISEVQR | 91 | 422-429 |
| and differentiation-enhancing factor 2 | | | | | | |
| (Pyk2 C-terminus associated | | | | | | |
| AT8B3_HUMAN (O60423) Probable | 4.37E−03 | 147935.6 | O60423 | QALMVTHKELATIK | 92 | 282-295 |
| phospholipid-transporting ATPase IK (EC | | | | | | |
| 3.6.3.1) (ATPase class I type | | | | | | |
| ST65G_HUMAN (O94864) STAGA | 3.26E−03 | 46163.76 | O94864 | YWGEIPISSSQTN | 93 | 6-30 |
| complex 65 gamma subunit | | | | RSSFDLLPREFR | | |
| (STAF65gamma) (SPTF-associated factor | | | | | | |
| 65 gamma) | | | | | | |
| ICT1_HUMAN (Q14197) Immature colon | 1.84E−04 | 23615.4 | Q14197 | FHLATAEWIAEPVRQKIAITHK | 94 | 103-124 |
| carcinoma transcript 1 protein | | | | | | |
| precursor (Digestion | | | | | | |
| substraction | | | | | | |
| FATH_HUMAN (Q14517) Cadherin-related | 6.63E−03 | 505962.8 | Q14517 | EVHSEIIQVEATDK | 95 | 836-849 |
| tumor suppressor homolog precursor (Fat | | | | | | |
| protein homolog) | | | | | | |
| Q59FQ9 (Q59FQ9) DDX19-like protein | 1.07E−03 | 35593.43 | Q59FQ9 | AGFAFEIPMKITWVSTVERGQK | 96 | 21-42 |
| variant (Fragment) | | | | | | |
| HHCM_HUMAN (Q05877) Hepatocellular | 4.42E−04 | 52117.47 | Q05877 | VIIISILQQVMANTLEINGK | 97 | 390-409 |
| carcinoma protein HHCM (HHC(M)) | | | | | | |
| Q5VW08 (Q5VW08) | 2.59E−03 | 116866.8 | Q5VW08 | FELQDSGSSLLPKEIVKVEK | 98 | 362-381 |
| OTTHUMP00000018324 (Hypothetical | | | | | | |
| protein KIAA0564) | | | | | | |
| Q8N8D3 (Q8N8D3) Hypothetical protein | 9.18E−03 | 53760.11 | Q8N8D3 | ELKALEEALRASQEK | 99 | 87-101 |
| FLJ39642 | | | | | | |
| Q8WZ24 (Q8WZ24) Hypothetical protein | 2.81E−03 | 32096.25 | Q8WZ24 | GCWGLSCQLLEHAVRLCR | 100 | 270-287 |
| EVPL_HUMAN (Q92817) Envoplakin (210 kDa | 9.32E−03 | 231475.4 | Q92817 | SLLEEER | 101 | 1157-1163 |
| paraneoplastic pemphigus antigen) | | | | | | |
| (p210) (210 kDa cornified | | | | | | |
| Q96RG5 (Q96RG5) Insulin receptor | 9.03E−03 | 137347.9 | Q96RG5 | LEYYESEKKWR | 102 | 75-85 |
| substrate 2 insertion mutant (Fragment) | | | | | | |
| PCDGM_HUMAN (Q9Y5F6) Protocadherin | 9.32E−03 | 101858 | Q9Y5F6 | SNTLRER | 103 | 812-818 |
| gamma C5 precursor (PCDH-gamma-C5) | | | | | | |
| Q4VXF3 (Q4VXF3) Transcription | 9.75E−03 | 100648 | Q4VXF3 | QLQEFIPNIKDR | 104 | 497-508 |
| termination factor, RNA polymerase I | | | | | | |
| (Fragment) | | | | | | |
| FBXL4_HUMAN (Q9UKA2) F-box/LRR- | 7.48E−03 | 70051.23 | Q9UKA2 | WEILWSERPTK | 105 | 172-182 |
| repeat protein 4 (F-box and | | | | | | |
| leucine-rich repeat protein 4) | | | | | | |
| (F-box pr | | | | | | |
| IGF1A_HUMAN (P01343) Insulin-like | 5.10E−03 | 17014.34 | P01343 | APQTGIVDECCFR | 106 | 86-98 |
| growth factor IA precursor (IGF-IA) | | | | | | |
| (Somatomedin C) (Mechano grow | | | | | | |
| KV1R_HUMAN (P01610) Ig kappa chain | 2.86E−06 | 11832.84 | P01610 | RLIYGATSLQSGVPSR | 107 | 46-61 |
| V-I region WEA | | | | | | |
| ANGI_HUMAN (P03950) Angiogenin | 2.32E−04 | 16539.44 | P03950 | NVVVACENGLPVHLDQSIFR | 108 | 126-145 |
| precursor (EC 3.1.27.—) | | | | | | |
| (Ribonuclease 5) | | | | | | |
| (RNase 5) | | | | | | |
| PLAK_HUMAN (P14923) Junction | 6.43E−04 | 81446.77 | P14923 | LNTIPLFVQLLYSSVENIQR | 109 | 581-600 |
| plakoglobin (Desmoplakin III) | | | | | | |
| AKT2_HUMAN (P31751) RAC-beta | 6.72E−04 | 55733.18 | P31751 | DIKLENLMLDKDGHIK | 110 | 275-290 |
| serine/threonine-protein kinase (EC | | | | | | |
| 2.7.1.37) (RAC-PK-beta) (Protein k | | | | | | |
| K1C20_HUMAN (P35900) Keratin, type I | 7.26E−03 | 48456.98 | P35900 | VFDDLTLHKTDLEIQIEELNK | 111 | 179-199 |
| cytoskeletal 20 (Cytokeratin-20) | | | | | | |
| (CK-20) (Keratin-20) (K20) (P | | | | | | |
| FAL39_HUMAN (P49913) Antibacterial | 2.44E−03 | 19289.16 | P49913 | SSDANLYR | 112 | 50-57 |
| protein FALL-39 precursor (FALL-39 | | | | | | |
| peptide antibiotic) (Cationic | | | | | | |
| PRELP_HUMAN (P51888) Prolargin | 3.74E−03 | 43782.22 | P51888 | ISSVPAINNR | 113 | 303-312 |
| precursor (Proline-arginine-rich end | | | | | | |
| leucine-rich repeat protein) | | | | | | |
| CUL4A_HUMAN (Q13619) Cullin-4A (CUL- | 8.78E−03 | 76771.75 | Q13619 | PLIACVEKQLLGEHLTAILQK | 114 | 185-205 |
| 4A) | | | | | | |
| DYH5_HUMAN (Q8TE73) Ciliary dynein | 8.02E−04 | 528667.8 | Q8TE73 | ESRNELQITSLNHK | 115 | 3618-3631 |
| heavy chain 5 (Axonemal beta dynein | | | | | | |
| heavy chain 5) (HL1) | | | | | | |
| NRX2A_HUMAN (Q9P2S2) Neurexin-2- | 4.70E−03 | 184864.2 | Q9P2S2 | SLQLSVDNVTVEGQMAGAHMR | 116 | 834-854 |
| alpha precursor (Neurexin II-alpha) | | | | | | |
| ST1C1_HUMAN (O00338) | 9.77E−03 | 34857.38 | O00338 | SILDQSISSFMR | 117 | 247-258 |
| Sulfotransferase 1C1 (EC 2.8.2.—) | | | | | | |
| (SULT1C#1) (ST1C2) (humSULTC2) | | | | | | |
| DHCA_HUMAN (P16152) Carbonyl | 1.73E−03 | 30224.83 | P16152 | IGVTVLSRIHAR | 118 | 198-209 |
| reductase [NADPH] 1 (EC 1.1.1.184) | | | | | | |
| (NADPH-dependent carbonyl reductase | | | | | | |
| RM28_HUMAN (Q13084) 39S ribosomal | 3.58E−03 | 33841.77 | Q13084 | EFYSEILDKKFTVTVTMR | 119 | 144-161 |
| protein L28, mitochondrial precursor | | | | | | |
| (L28mt) (MRP-L28) (Melanoma | | | | | | |
| UBR1_HUMAN (Q8IWV7) Ubiquitin-protein | 1.66E−03 | 200079.2 | Q8IWV7 | TVVQSCGHSLETK | 120 | 584-596 |
| ligase E3 component N-recognin-1 | | | | | | |
| (EC 6.—. —.—) (Ubiquitin-prot | | | | | | |
| SMYD1_HUMAN (Q8NB12) SET and | 8.58E−03 | 56579.95 | Q8NB12 | LKDDLFLGVKDNPK | 121 | 283-296 |
| MYND domain containing protein 1 | | | | | | |
| NOX1_HUMAN (Q9Y5S8) NADPH oxidase | 4.35E−03 | 64829.09 | Q9Y5S8 | QATDGSLASILSSLSHDEKK | 122 | 131-150 |
| homolog 1 (NOX-1) (NOH-1) | | | | | | |
| (NADH/NADPH mitogenic oxidase subunit P | | | | | | |
| O75229 (O75229) R31449_3 (Fragment) | 6.40E−03 | 93393.15 | O75229 | FNIFYPDLIDK | 123 | 556-566 |
| Q6ZTY8 (Q6ZTY8) Hypothetical protein | 2.80E−03 | 135743.9 | Q6ZTY8 | LTLARSLVLLDDLTKAEK | 124 | 902-919 |
| FLJ44112 | | | | | | |
| TRPC4_HUMAN (Q9UBN4) Short transient | 1.61E−04 | 112030.3 | Q9UBN4 | VCPFKSEKVVVEDTVPIIPK | 125 | 927-946 |
| receptor potential channel 4 (TrpC4) | | | | | | |
| (trp-related protein 4) ( | | | | | | |
| BPAEA_HUMAN (O94833) Bullous | 6.56E−03 | 590630.3 | O94833 | QFHEAWSKLMEWLEESEK | 126 | 4296-4313 |
| pemphigoid antigen 1, isoforms 6/9/10 | | | | | | |
| (Trabeculin-beta) (Bullous pemph | | | | | | |
| K1C17_HUMAN (Q04695) Keratin, type I | 1.37E−09 | 47945.07 | Q04695 | YCVQLSQIQGLI | 127 | 334-356 |
| cytoskeletal 17 (Cytokeratin-17) | | | | GSVEEQLAQLR | | |
| (CK-17) (Keratin-17) (K17) (3 | | | | | | |
| Q59FP4 (Q59FP4) Integrin-linked kinase | 8.99E−04 | 13981.02 | Q59FP4 | TSVQQTLPLRHPLP | 128 | 8-31 |
| variant (Fragment) | | | | TLTRLYLASR | | |
| Q6XQN6 (Q6XQN6) Nicotinate | 3.83E−03 | 60239.51 | Q6XQN6 | LDSGDLLQQAQEIR | 129 | 319-332 |
| phosphoribosyltransferase-like protein | | | | | | |
| Q6ZTY7 (Q6ZTY7) Hypothetical protein | 5.85E−03 | 57512.96 | Q6ZTY7 | MARIILQDEDVTTKIDNDWK | 130 | 177-196 |
| FLJ44113 | | | | | | |
| CM35H_HUMAN (Q9UGN4) CMRF35-H | 6.75E−03 | 33151.84 | Q9UGN4 | EVEVEYSTVASPR | 131 | 250-262 |
| antigen precursor (CMRF35-H9) (CMRF- | | | | | | |
| 35-H9) (Inhibitory receptor prote | | | | | | |

In addition, the tandem mass spectra were analyzed using more stringent filtering criteria, with a goal of reducing false positives. In particular, the spectra were analyzed using the filtering algorithms of the Scalfold Software (Proteome Software Inc., Portland, Oreg.).

The results are provided in Table 2. In short, 86 peptides were identified as biomarkers that correlate to the disease state. Thus, evaluating patient samples for the presence of one or more of these biomarkers will provide a useful method for detecting breast cancer.

TABLE 2

| | | | | | | |
|---|---|---|---|---|---|---|
| | | Protein | | | Calculated | |
| | Protein | molecular | | SEQ | Peptide | |
| | accession | weight | | ID | Mass | Residue |
| Protein name | numbers | (Da) | Peptide sequence | NOs | (AMU) | number |

| | | | | | | |
|---|---|---|---|---|---|---|
| Fibrinogen gamma chain | FIBG_HUMAN | 51495.3 | ASTPNGYDNGIIWATWK | 132 | 1893.914 | 383-399 |
| precursor | | | | | | |
| Complement factor B precursor | CFAB_HUMAN | 85515.2 | DFHINLFQVLPWLK | 133 | 1769.975 | 740-753 |
| (EC 3.4.21.47) (C3/C5 | | | | | | |
| convertase) (Properdin factor B) | | | | | | |
| (Glycine-rich beta glycoprotein) | | | | | | |
| (GBG) (PBF2) [Contains: | | | | | | |
| Complement factor B Ba | | | | | | |
| fragment; Complement factor B | | | | | | |
| Bb fragment] | | | | | | |
| Beta-2-glycoprotein I precursor | APOH_HUMAN | 38280.5 | TCPKPDDLPFSTVVPLK | 134 | 1914.005 | 22-38 |
| (Apolipoprotein H) (Apo-H) | | | | | | |
| (B2GPI) (Beta(2)GPI) (Activated | | | | | | |
| protein C-binding protein) (APC | | | | | | |
| inhibitor) (Anticardiolipin cofactor) | | | | | | |
| Alpha-fetoprotein precursor | FETA_HUMAN | 68660.2 | AENAVECFQTK | 135 | 1296.59 | 195-205 |
| (Alpha-fetoglobulin) (Alpha-1- | | | | | | |
| fetoprotein) | | | | | | |
| Hypothetical protein | Q6MZQ6 | 52024.1 | LSCAASGFTFR | 136 | 1216.579 | 39-49 |
| DKFZp686G11190 | | | | | | |
| Keratin 10 | Q14664 | 57231.3 | GSSGGGCFGGSSGG | 137 | 2342.985 | 53-79 |
| | | | YGGLGGFGGGSFR | | | |
| Apolipoprotein M (Apo-M) (ApoM) | APOM_HUMAN | 21235.9 | KWIYHLTEGSTDLR | 138 | 1718.887 | 99-112 |
| (G3a protein) | | | | | | |
| Gelsolin precursor (Actin- | GELS_HUMAN | 85679.8 | EVQGFESATFLGYFK | 139 | 1722.838 | 148-162 |
| depolymerizing factor) (ADF) | | | | | | |
| (Brevin) (AGEL) | | | | | | |
| Immunoglobulin J chain | IGJ_HUMAN | 15576.5 | SSEDPNEDIVER | 140 | 1389.614 | 25-36 |
| Kininogen-1 precursor (Alpha-2- | KNG1_HUMAN | 71927.5 | YNSQNQSNNQFVLYR | 141 | 1874.878 | 44-58 |
| thiol proteinase inhibitor) | | | | | | |
| [Contains: Kininogen-1 heavy | | | | | | |
| chain; Bradykinin (Kallidin I); | | | | | | |
| Lysyl-bradykinin (Kallidin II); | | | | | | |
| Kininogen-1 light chain; Low | | | | | | |
| molecular weight growth | | | | | | |
| promoting factor] | | | | | | |
| GMP synthase [glutamine- | GUAA_HUMAN | 76698.7 | LYGAQFHPEVGLTENGK | 142 | 1859.929 | 184-200 |
| hydrolyzing] (EC 6.3.5.2) | | | | | | |
| (Glutamine amidotransferase) | | | | | | |
| (GMP synthetase) | | | | | | |
| 1-phosphatidylinositol-4,5- | PLCB3_HUMAN | 138784.7 | LVAGQQQVLQQLAEEEPK | 143 | 2008.072 | 1150-1167 |
| bisphosphate phosphodiesterase | | | | | | |
| beta 3 (EC 3.1.4.11) | | | | | | |
| (Phosphoinositide phospholipase | | | | | | |
| C) (PLC-beta-3) (Phospholipase | | | | | | |
| C-beta-3) | | | | | | |
| Hypothetical protein FLJ45653 | Q6ZSB9 | 85057.6 | QAPDTSDGSCTELPFK | 144 | 1752.775 | 193-208 |
| Zinc finger protein 157 (HZF22) | ZN157_HUMAN | 58272 | IQTLDQNVEYNGCR | 145 | 1709.792 | 127-140 |
| Biogenesis of lysosome-related | BL1S1_HUMAN | 14293.3 | KELQEKR | 146 | 930.5373 | 17-23 |
| organelles complex-1, subunit 1 | | | | | | |
| (BLOC-1 subunit 1) (GCN5-like | | | | | | |
| protein 1) (RT14 protein) | | | | | | |
| Fibrinogen beta chain precursor | FIBB_HUMAN | 55910.6 | REEAPSLRPAP | 147 | 2107.105 | 53-72 |
| [Contains: Fibrinopeptide B] | | | PPISGGGYR | | | |
| Insulin-like growth factor binding | Q6P1M6 | 31656.1 | FLNVLSPR | 148 | 945.5522 | 226-233 |
| protein 3 | | | | | | |
| C18orf34 protein | Q5BJE1 | 100177.3 | LTEDNKKLEIDINK | 149 | 1672.913 | 449-462 |
| Zinc finger and BTB domain | ZBTB1_HUMAN | 81998.7 | MDLEENPDEQSEIR | 150 | 1704.739 | 488-501 |
| containing protein 1 | | | | | | |
| Nuclear factor erythroid 2 related | NF2L2_HUMAN | 67809.4 | EKGENDK | 151 | 819.3849 | 542-548 |
| factor 2 (NF-E2 related factor 2) | | | | | | |
| (NFE2-related factor 2) (Nuclear | | | | | | |
| factor, erythroid derived 2, like 2) | | | | | | |
| (HEBP1) | | | | | | |
| Receptor interacting protein | Q5RKT0 | 100004.6 | QNLRETQKFFR | 152 | 1466.787 | 43-53 |
| kinase 5, isoform 2 | | | | | | |
| Nipped-B-like protein (Delangin) | NIPBL_HUMAN | 316037.3 | ELPPELLAEIESTMPLCER | 153 | 2227.099 | 1050-1068 |
| (SCC2 homolog) | | | | | | |
| Thrombospondin-2 precursor | TSP2_HUMAN | 129934.9 | MVWRLVLLALWVW | 154 | 2734.466 | 1-23 |
| | | | PSTQAGHQDK | | | |
| Alpha-1-acid glycoprotein 2 | A1AG2_HUMAN | 23585.2 | EQLGEFYEALDCLCIPR | 155 | 2112.974 | 154-170 |
| precursor (AGP 2) (Orosomucoid- | | | | | | |
| 2) (OMD 2) | | | | | | |
| Apolipoprotein B-100 precursor | APOB_HUMAN | 515554.3 | VELEVPQLCSFILK | 156 | 1674.914 | 80-93 |
| (Apo B-100) [Contains: | | | | | | |
| Apolipoprotein B-48 (Apo B-48)] | | | | | | |
| Hypothetical protein FLJ44261 | Q6ZTS6 | 109100.2 | APEPDLMSPTR | 157 | 1213.589 | 821-831 |
| CMRF35-H antigen precursor | CM35H_HUMAN | 33154.7 | EVEVEYSTVASPR | 158 | 1465.717 | 250-262 |
| (CMRF35-H9) (CMRF-35-H9) | | | | | | |
| (Inhibitory receptor protein 60) | | | | | | |
| (IRp60) (IRC1/IRC2) (NK | | | | | | |
| inhibitory receptor) | | | | | | |
| Two-pore calcium channel protein | Q8NHX9 | 85283.9 | SYGSVLLSAEEFQK | 159 | 1557.78 | 385-398 |
| 2 (Two pore segment channel 2) | | | | | | |
| Protocadherin beta 14 precursor | PCDBE_HUMAN | 87532.2 | DINDHSPTFLDK | 160 | 1401.665 | 125-136 |
| (PCDH-beta14) | | | | | | |
| Dimethylglycine dehydrogenase, | M2GD_HUMAN | 96790.9 | LNKPADFIGK | 161 | 1102.626 | 748-757 |
| mitochondrial precursor (EC | | | | | | |
| 1.5.99.2) (ME2GLYDH) | | | | | | |
| Angiopoietin-1 receptor precursor | TIE2_HUMAN | 125795 | FNPICKASGWP | 162 | 2532.263 | 366-387 |
| (EC 2.7.1.112) (Tyrosine-protein | | | LPTNEEMTLVK | | | |
| kinase receptor TIE-2) (Tyrosine- | | | | | | |
| protein kinase receptor TEK) | | | | | | |
| (P140 TEK) (Tunica interna | | | | | | |
| endothelial cell kinase) (CD202b | | | | | | |
| antigen) | | | | | | |
| MGC5297 protein (Fragment) | Q9BVD3 | 62640.1 | VLTPYCYTIDVEIK | 163 | 1713.878 | 446-459 |
| Dystrobrevin beta (Beta- | DTNB_HUMAN | 71338.1 | LIARYAARLAAEAGNVTR | 164 | 1916.083 | 404-421 |
| dystrobrevin) (DTN-B) | | | | | | |
| A-kinase anchor protein 3 | AKAP3_HUMAN | 94719.5 | SCDASLAELGDDKSGDASR | 165 | 1953.846 | 687-705 |
| (Protein kinase A anchoring | | | | | | |
| protein 3) (PRKA3) (A-kinase | | | | | | |
| anchor protein 110 kDa) (AKAP | | | | | | |
| 110) (Sperm oocyte binding | | | | | | |
| protein) (Fibrousheathin I) | | | | | | |
| (Fibrous sheath protein of 95 kDa) | | | | | | |
| (FSP95) | | | | | | |
| Netrin receptor UNC5C precursor | UNC5C_HUMAN | 103084.7 | VYEMYVTVHR | 166 | 1296.641 | 564-573 |
| (Unc-5 homolog C) (Unc-5 | | | | | | |
| homolog 3) | | | | | | |
| EPM2A-interacting protein 1 | EPMIP_HUMAN | 70352.8 | ILSIDRNLRNQLFNR | 167 | 1872.057 | 154-168 |
| (Laforin-interacting protein) | | | | | | |
| Transmembrane protein 2 | Q9UHN6 | 154359 | YVGTGGIDQK | 168 | 1037.527 | 847-856 |
| Symplekin | SYMPK_HUMAN | 141135.5 | NMPSSKDTRK | 169 | 1163.584 | 338-347 |
| Hook homolog 3 (hHK3) | HOOK3_HUMAN | 83110 | RAIIEDLEPR | 170 | 1211.675 | 566-575 |
| Hypothetical protein SERAC1 | Q6PKF3 | 74145.6 | KDAFLYQRTLQFIR | 171 | 1798.997 | 632-645 |
| 39S ribosomal protein L28, | RM28_HUMAN | 33846.3 | EFYSEILDKKFTVTVTMR | 172 | 2207.142 | 144-161 |
| mitochondrial precursor (L28mt) | | | | | | |
| (MRP-L28) (Melanoma antigen | | | | | | |
| p15) (Melanoma-associated | | | | | | |
| antigen recognized by T | | | | | | |
| lymphocytes) | | | | | | |
| Seven transmembrane helix | Q8NGB0 | 156486.6 | WLSSPVFSLRR | 173 | 1347.754 | 334-344 |
| receptor | | | | | | |
| Lymphotactin precursor (XCL1) | XCL1_HUMAN | 12498.7 | AVIFITKRGLK | 174 | 1245.805 | 57-67 |
| (Cytokine SCM-1) (ATAC) | | | | | | |
| (Lymphotaxin) (SCM-1-alpha) | | | | | | |
| (Small inducible cytokine C1) (XC | | | | | | |
| chemokine ligand 1) | | | | | | |
| Peptidoglycan recognition protein | PGRP_HUMAN | 21712.6 | VPTPQAIRAAQGLLA | 175 | 3122.726 | 139-168 |
| precursor (PGRP-S) | | | CGVAQGALRSNYVLK | | | |
| Ankyrin repeat domain protein | ANR18_HUMAN | 115652.2 | NQANIHAVDNFK | 176 | 1370.682 | 189-200 |
| 18A | | | | | | |
| Mitochondrial ribosomal protein | Q9BYD0 | 28446.1 | VLSPYDLTHK | 177 | 1172.632 | 229-238 |
| L16 (L16mt) | | | | | | |
| Protein disulfide-isomerase A4 | PDIA4_HUMAN | 72916 | NNKGPVKVVVGK | 178 | 1238.759 | 522-533 |
| precursor (EC 5.3.4.1) (Protein | | | | | | |
| ERp-72) (ERp72) | | | | | | |
| 2-5A-dependent ribonuclease | RN5A_HUMAN | 83516.7 | RGANVNLR | 179 | 899.5175 | 147-154 |
| (EC 3.1.26.—) (2-5A-dependent | | | | | | |
| RNase) (Ribonuclease L) (RNase | | | | | | |
| L) (Ribonuclease 4) | | | | | | |
| WD-repeat protein 75 | WDR75_HUMAN | 94483 | SEQPTLVTASKDGYFK | 180 | 1770.892 | 456-471 |
| Zinc finger protein 294 | ZN294_HUMAN | 200525.3 | VFKMLLGDEKQSIVQAK | 181 | 1934.079 | 629-645 |
| Hypothetical protein FLJ44356 | Q6ZTQ6 | 26752.7 | GADPPPPPSRTGR | 182 | 1304.671 | 59-71 |
| Protein C6orf149 | CF149_HUMAN | 10740.9 | ENKNVKDPVEIQTLVNK | 183 | 1968.077 | 42-58 |
| Ubiquitin specific protease 48 | Q5SZI4 | 18677.1 | FNDEDIEKMEGKK | 184 | 1582.743 | 47-59 |
| Intercellular adhesion molecule 3 | ICAM3_HUMAN | 59363.8 | ADQEGAREIVC | 185 | 2230.1 | 284-303 |
| precursor (ICAM-3) (ICAM-R) | | | NVTLGGERR | | | |
| (CDw50) (CD50 antigen) | | | | | | |
| Hypothetical protein | Q86VG6 | 11513.3 | AGECVTAGGLGGARRR | 186 | 1587.814 | 12-27 |
| Zinc finger protein HRX (ALL-1) | HRX_HUMAN | 431732.3 | QVSQPALVIPP | 187 | 2080.156 | 1297-1316 |
| (Trithorax-like protein) | | | QPPTTGPPR | | | |
| Hypothetical protein FLJ37300 | Q52M87 | 61958.1 | HLLTIAGWKHEK | 188 | 1432.807 | 457-468 |
| Insulin-like growth factor IA | IGF1A_HUMAN | 17008.1 | APQTGIVDECCFR | 189 | 1552.689 | 86-98 |
| precursor (IGF-IA) (Somatomedin | | | | | | |
| C) (Mechano growth factor) | | | | | | |
| (MGF) | | | | | | |
| Guanine nucleotide exchange | MCF2L_HUMAN | 123968.6 | FKPMQRHLFLHEK | 190 | 1710.927 | 870-882 |
| factor DBS (DBL's big sister) | | | | | | |
| (MCF2 transforming sequence- | | | | | | |
| like protein) (Fragment) | | | | | | |
| Hypothetical protein FLJ40235 | Q8N7X8 | 21265.9 | VRASQELEMSLK | 191 | 1390.736 | 160-171 |
| Novel protein | Q9H4G2 | 164839.6 | EKLSLEPVLPARNPNR | 192 | 1833.035 | 309-324 |
| Chromosome 9 open reading | Q5SZB4 | 47622.5 | EDPDFLGAFLGELLPSR | 193 | 1875.95 | 134-150 |
| frame 50 | | | | | | |
| Piccolo protein (Aczonin) | PCLO_HUMAN | 566639.4 | VMSDGPVKPEGAK | 194 | 1314.673 | 4843-4855 |
| Hypothetical protein FLJ90556 | Q8N2J3 | 30614.7 | DSGGQTSAGCPSGWLGTR | 195 | 1793.788 | 123-140 |
| Prokineticin receptor 2 (PK-R2) | PKR2_HUMAN | 43979 | YLAIVHPLKPR | 196 | 1306.8 | 154-164 |
| (G-protein coupled receptor 73- | | | | | | |
| like 1) (GPR73b) (GPRg2) | | | | | | |
| 1-phosphatidylinositol-4,5- | PLCB2_HUMAN | 133665.8 | QAACLEQIREMEK | 197 | 1605.773 | 1101-1113 |
| bisphosphate phosphodiesterase | | | | | | |
| beta 2 (EC 3.1.4.11) | | | | | | |
| (Phosphoinositide phospholipase | | | | | | |
| C) (PLC-beta-2) (Phospholipase | | | | | | |
| C-beta-2) | | | | | | |
| Reticulon-4 (Neurite outgrowth | RTN4_HUMAN | 129916.6 | LSALPPEGGKPYLE | 198 | 2604.393 | 788-811 |
| inhibitor) (Nogo protein) (Foocen) | | | SFKLSLDNTK | | | |
| (Neuroendocrine-specific protein) | | | | | | |
| (NSP) (Neuroendocrine-specific | | | | | | |
| protein C homolog) (RTN-x) | | | | | | |
| (Reticulon-5) | | | | | | |
| Tripartite motif protein 8 (RING | TRIM8_HUMAN | 61469.3 | QTVEVLDK | 199 | 931.5102 | 249-256 |
| finger protein 27) (Glioblastoma- | | | | | | |
| expressed RING finger protein) | | | | | | |
| Beta-adducin (Erythrocyte | ADDB_HUMAN | 80836.4 | TTWMKADEVEK | 200 | 1337.641 | 460-470 |
| adducin beta subunit) | | | | | | |
| Hypothetical protein FLJ13755 | Q9H8C8 | 148979 | FDLKQWLSATK | 201 | 1336.727 | 601-611 |
| Elastin precursor (Tropoelastin) | ELN_HUMAN | 68478.6 | FPGVGVLPGVPTGAGVK | 202 | 1551.89 | 160-176 |
| Hypothetical protein PRSS35 | Q8N3Z0 | 47052.9 | YAQICLWIHGNDANCAYG | 203 | 2125.922 | 396-413 |
| (ENML522) | | | | | | |
| Diacylglycerol kinase, beta (EC | DGKB_HUMAN | 90578.9 | CLRWGGGYEGENLMK | 204 | 1769.811 | 532-546 |
| 2.7.1.107) (Diglyceride kinase) | | | | | | |
| (DGK-beta) (DAG kinase beta) | | | | | | |
| (90 kDa diacylglycerol kinase) | | | | | | |
| Hypothetical protein | Q6PJ41 | 3537.6 | MIMILVLLSLGR | 205 | 1358.827 | 1-12 |
| Zinc finger protein 592 | ZN592_HUMAN | 137535.7 | GSDLPPDPHNCGK | 206 | 1393.617 | 97-109 |
| Envoplakin (210 kDa | EVPL_HUMAN | 231600.4 | SLLEEER | 207 | 875.4474 | 1157-1163 |
| paraneoplastic pemphigus | | | | | | |
| antigen) (p210) (210 kDa | | | | | | |
| cornified envelope precursor | | | | | | |
| protein) | | | | | | |
| ATP-binding cassette sub-family | ABCA2_HUMAN | 269960.8 | ARRFLWNLILDLIK | 208 | 1771.075 | 2221-2234 |
| A member 2 (ATP-binding | | | | | | |
| cassette transporter 2) (ATP- | | | | | | |
| binding cassette 2) | | | | | | |
| Putative helicase Mov10l1 (EC | M10L1_HUMAN | 135276.7 | RFNVAITR | 209 | 976.5691 | 1131-1138 |
| 3.6.1.—) (Moloney leukemia virus | | | | | | |
| 10-like protein 1) (MOV10-like 1) | | | | | | |
| Novel protein (MGC26989) | Q5T1M9 | 62944.4 | PLAIAKQASFSSK | 210 | 1347.764 | 279-291 |
| Tic | O95621 | 116328.1 | SMSCQEFITNLNGLR | 211 | 1769.832 | 702-716 |
| Hypothetical protein FLJ46306 | Q6ZRJ5 | 18924.6 | EKEQEAGGVSYWDIGK | 212 | 1795.851 | 35-50 |
| Hypothetical class II basic helix- | Q7RTU2 | 32314.4 | RQRGDAGSPWGCPLCPDR | 213 | 2084.951 | 151-168 |
| loop-helix protein MESP2 | | | | | | |
| (Fragment) | | | | | | |
| RSK-like protein (Ribosomal | Q8TDD3 | 118668.4 | RNPEDVQEIIVWKR | 214 | 1781.966 | 40-53 |
| protein S6 kinase, 52 kDa, | | | | | | |
| polypeptide 1) | | | | | | |
| Histone H2B-related protein (H2B | Q7Z2G1 | 19581.4 | LAESEGTK | 215 | 834.421 | 153-160 |
| histone family, member W, testis- | | | | | | |
| specific) | | | | | | |
| LOC493860 protein | Q6P5Q7 | 45467 | FLNLQNEHEKALGTWKR | 216 | 2084.104 | 237-253 |
| Glutamine synthetase (EC | GLNA_HUMAN | 41915.7 | TCLLNETGDEPFQYK | 217 | 1814.827 | 357-371 |
| 6.3.1.2) (Glutamate--ammonia | | | | | | |
| ligase) (GS) | | | | | | |

### Example 2

### Cancer-Related Peptides are not Necessarily Biomarkers

The above methods showed that a number of peptides previously known to be associated with breast cancer were not indicative of a disease state, and, thus, not useful as a biomarker. Examples include, RUN and FYVE domain-containing 1 variant (fragment), haptoglobin precursor that contains: haptoglobin alpha chain and haptoglobin beta chain, tetranectin precursor (TN) (Plasminogen-kringle 4 binding protein), vitamin D-binding protein, apolipoprotein C-IV precursor (Apo-CIV or ApoC-IV), VH1 protein precursor (fragment), Ig kappa chain V-III region SIE, hypothetical protein Q569I7, haptoglobin-related protein precursor, breast carcinoma amplified sequence 1 (novel amplified in breast cancer 1) (amplified and overexpressed in breast cancer), sodium-D-glucose cotransporter (regulatory solute carrier protein, family 1, member 1), complement component C8 gamma chain precursor, Ig heavy chain V-III region TIL, hypothetical protein DKFZp686I04196 (fragment), alpha-2-macroglobulin precursor (Alpha-2-M), hypothetical protein DKFZp761P18121, fibrinogen alpha chain precursor that contains fibrinopeptide A, IGHG1 protein, hypothetical protein MGC27016, seprase (EC 3.4.21.-) (fibroblast activation protein alpha) (integral membrane serine protease) (170-kDa melanoma membrane-bound gelatinase), PTPL1-associated RhoGAP, zinc finger protein 385 (hematopoietic zinc finger protein) (retinal zinc finger protein), hypothetical protein FLJ45950, hypothetical protein FLJ39462, smoothelin, latrophilin-3 precursor (calcium-independent alpha-latrotoxin receptor 3) (lectomedin-3), type III iodothyronine deiodinase (EC 1.97.1.11) (type-III 5′deiodinase) (DIOIII) (type 3 DI) (5DIII).

We claim:

1. A method for detecting breast cancer in a patient, comprising:
(i) obtaining a biological sample from said patient; and
(ii) evaluating said sample or a fraction of said sample for the presence of at least one biomarker selected from the group of peptides having the amino acid sequences of SEQ ID NOs: 132-217,
wherein the presence of said at least one biomarker is indicative of breast cancer.

2. The method according to claim 1, further comprising, prior to the evaluation step, harvesting low molecular weight peptides from said sample to generate at least one fraction comprising said peptides.

3. The method according to claim 1, wherein said biological sample is blood, serum or plasma.

4. The method according to claim 1, wherein the evaluation step comprises an assay selected from the group consisting of mass spectrometry, immunoassay, immuno-mass spectrometry and suspension bead array.

5. The method according to claim 4, wherein said immunoassay is an enzyme linked immunosorbent assay or ELISA.

6. The method according to claim 4, wherein said mass spectrometry comprises multiple reaction monitoring (MRM).

7. The method according to claim 2, further comprising digesting said low molecular weight peptides.

8. The method according to claim 7, wherein said digestion comprises a trypsin digestion.

9. The method according to claim 1, wherein the breast cancer is in an early stage.

10. The method according to claim 1, wherein the breast cancer is in stage T1a.

11. The method according to claim 1, wherein said evaluation step comprises evaluating said sample for the presence of at least biomarkers having the amino acid sequences of SEQ ID NOs: 132, 139, 141 and 148.

12. A method for monitoring the progression of breast cancer in a patient, comprising:
(i) obtaining a biological sample from said patient;
(ii) evaluating said sample or a fraction of said sample for the presence of at least one biomarker selected from the group of peptides having the amino acid sequences of SEQ ID NOs: 132-217, wherein the presence of said at least one biomarker is indicative of breast cancer; and optionally
(iii) repeating steps (i) and (ii).

13. The method according to claim 12 further comprising, prior to the evaluation step, harvesting low molecular weight peptides from said sample to generate at least one fraction comprising said peptides.

14. The method according to claim 12, wherein said evaluation step comprises evaluating said sample for the presence of at least biomarkers having the amino acid sequences of SEQ ID NOs: 132, 139, 141 and 148.

15. An antibody specific for a peptide selected from the group of peptides having the amino acid sequences of SEQ ID NOs: 132-217.

16. The antibody according to claim 15, wherein said antibody is a monoclonal antibody.

17. The antibody according to claim 15, wherein said antibody is a polyclonal antibody.

18. The antibody according to claim 15, wherein said antibody is a chimeric antibody.

19. The antibody according to claim 15, wherein the peptide is selected from the group of peptides having the amino acid sequences of SEQ ID NOs: 132, 139, 141 and 148.

20. A kit for detecting breast cancer in a patient, comprising at least one antibody according to claim 15.
